(19) United States
(12) Patent Application Publication
(10) Pub. No.: US 2023/0068307 A1
(43) Pub. Date: Mar. 2, 2023

(54) Joint Motion Measurement Apparatus and Method of Use

(71) Applicant: The Curators of the University of Missouri, Columbia, MO (US)

(72) Inventors: Trent M. Guess, Columbia, MO (US); James L. Cook, Columbia, MO (US); James P. Stannard, Columbia, MO (US); Jamie B. Hall, Columbia, MO (US)

(21) Appl. No.: 17/797,038

(22) PCT Filed: Feb. 5, 2021
(86) PCT No.: PCT/US2021/016872

§ 371 Date: Aug. 2, 2022

Related U.S. Application Data
(60) Provisional application No. 62/971,704, filed on Feb. 7, 2020.

(51) Int. Cl. A61B 5/11; A61B 5/00; A61B 5/107

(52) U.S. Cl. CPC A61B 5/1121; A61B 5/6812; A61B 5/1114; A61B 5/1107; A61B 5/4585; A61B 5/7217; A61B 5/4519; A61B 5/1071; A61B 2562/0219; A61B 2562/04

(57) ABSTRACT

A joint motion measurement apparatus includes securing mechanisms that secure sensors to various body parts such as the leg, including the femur, tibia, malleoli and/or calcaneus. The sensors are configured to measure a position and/or motion of the various parts of the leg relative to one another. The sensor data is usable to determine kinematic and/or muscle properties of the leg including knee laxity, tibiofemoral measurements and/or spastic properties.

### CROSS-REFERENCE TO RELATED APPLICATIONS

This application claims priority to U.S. provisional application Ser. No. 62/971,704, filed on Feb. 7, 2020, the entirety of which is incorporated by reference herein.

### STATEMENT REGARDING FEDERALLY SPONSORED RESEARCH OR DEVELOPMENT

Not Applicable.

### APPENDIX

Not Applicable.

### BACKGROUND

Accurate assessment of three-dimensional (3D) joint kinematics is critical for screening, diagnostic, and therapeutic applications in orthopaedics. Such kinematics may include arthrokinematics and osteokinematics, which may further be analyzed in terms of tibiofemoral kinematics. However, relative motion between bone and skin reduces the accuracy of marker-based tibiofemoral kinematic measures. While intracortical pins and biplane imaging can provide precise in vivo bone motion, these options are restricted by cost, availability, patient acceptance and/or ethical considerations. As such, information on tibiofemoral kinematics during knee movement and other functional weight-bearing activities is limited.

Knowledge of 3D motion of the knee (e.g., tibia relative to femur) would aid in diagnosis and treatment of knee related impairment and would benefit understanding, for example, of the influence of gait impairment on knee motion, mechanisms of ligament injury, outcomes of total knee arthroplasty, and effects of assistive devices (e.g., ankle foot orthotics). Such knee related impairment includes, for example, impairment resulting from disorders such as cerebral palsy and the like, impairment resulting from other neurological conditions such as stroke and the like, and impairment from musculoskeletal disorders and the like. Such impairment may include, for example, muscle spasticity, joint degeneration, osteoarthritis and general knee pain.

### SUMMARY OF THE INVENTION

This section provides a general summary of the disclosure, and is not a comprehensive disclosure of its full scope or all of its features.

There is a need to accurately monitor and characterize in vivo joint motion, such as knee motion, in a non-invasive, cost-effective manner. An aspect of the invention provides a joint arthrometer such as a knee arthrometer for measuring knee laxity. The knee arthrometer includes a femoral frame and a tibial frame. The femoral frame is attachable to a leg about a distal femur. The distal femur and the leg both have a medial side, a lateral side, an anterior side, a posterior side, a lateral epicondyle, and a medial epicondyle. The femoral frame includes a first arm portion, a second arm portion, a connecting portion, a femoral motion sensor, and a femoral securing mechanism, and may also include additional femoral arm portions. The first arm portion is configured to contact the leg about the lateral epicondyle, and the second arm portion is configured to contact the leg about the medial epicondyle. The connecting portion connects the first and second arm portions. The femoral motion sensor is coupled to the femoral frame and is configured to measure motion with six degrees of freedom and output femoral motion data associated with the measured motion. The femoral arm portions may contact portions of the leg above the first and second arm portions. The tibial frame is attachable to the leg about a tibia. The tibia has a proximal end, a distal end, and an anterior crest. The tibial frame is attachable to the leg about the tibia closer to the proximal end of the tibia than the distal end of the tibia. The tibial frame includes a tibial securing mechanism, a rigid frame portion, and a tibial motion sensor. The tibial motion sensor is coupled to the rigid frame portion and configured to measure motion with six degrees of freedom and output tibial motion data associated with the measured motion. The femoral motion data and the tibial motion data are usable, in conjunction, to determine knee laxity and/or tibiofemoral rotations during movement (e.g., step-up and squatting tasks).

Another aspect of the invention provides a method for measuring knee laxity. The method includes attaching a femoral frame to a leg about a distal femur. The distal femur and the leg both have a medial side, a lateral side, an anterior side, a posterior side, a lateral epicondyle, and a medial epicondyle. The femoral frame includes a first arm portion, a second arm portion, a connecting portion, a femoral motion sensor, and a femoral securing mechanism. The first arm portion contacts the leg about the lateral epicondyle, and the second arm portion contacts the leg about the medial epicondyle. The connecting portion connects the first and second arm portions, and the connecting portion is positioned above the anterior side of the leg. The femoral motion sensor is coupled to the femoral frame and configured to measure motion with six degrees of freedom. Additionally, femoral arm portions may further couple the arthrometer to a leg of a test subject. The method further includes attaching a tibial frame to the leg about a tibia. The tibia has a proximal end and a distal end. The tibial frame is attached to the leg about the tibia closer to the proximal end of the tibia than the distal end of the tibia. The tibial frame includes a tibial securing mechanism, a rigid frame portion, and a tibial motion sensor. The tibial motion sensor is coupled to the rigid frame portion and configured to measure motion with six degrees of freedom. The tibial securing mechanism is attached to the rigid frame portion and positioned about the leg and tibia. The method still further includes moving the tibia relative to the femur in at least one cycle including knee flexion and extension, recording the motion of the femoral motion sensor and recording the motion of the tibial motion sensor through the at least one cycle, and determining knee laxity based on the recorded motion of the femoral and tibial motion sensors.

Yet another aspect of the invention provides for a knee arthrometer and method of efficiently and accurately measuring tibiofemoral bone motion on test subjects performing functional tasks, such as stepping, squatting, treadmill walking, lateral step-down, lunging, and hopping tasks, as well as on test subjects undergoing physical evaluations (e.g., from a physical therapist), such as spasticity evaluations, and also on test subjects wearing orthotics. Measuring tibiofemoral bone motion may include measuring motion of test subjects wearing ankle foot orthotics (e.g., such as a solid ankle foot orthotics prescribed to improve gait). Motion measurements may also be conducted with sensors placed at areas outside of the tibiofemoral region, such as the area near the malleoli and the area near the calcaneus. This method allows for differences in knee kinematics to be determined in real-time, based on calculations performed with respect to a (e.g., Cartesian) coordinate system. The knee arthrometer and associated software (e.g., custom algorithm) can be used together as a knee arthrometer testing system (KATS).

As discussed above, the present invention comprises a knee arthrometer that may include a femoral frame and a tibial frame. The femoral frame may be attachable to a leg about a distal femur and includes a first arm portion, a second arm portion, a connecting portion, and a femoral motion sensor. The first arm portion may be configured to contact the leg about the lateral epicondyle, and the second arm portion may be configured to contact the leg about the medial epicondyle. The femoral motion sensor may be configured to measure motion with six degrees of freedom and output femoral motion data. The tibial frame may include a tibial motion sensor and may be attachable to the leg about a tibia. The tibial motion may be configured to measure motion with six degrees of freedom and output tibial motion data associated with measured motion. The femoral motion data and the tibial motion data may be usable to determine knee laxity and/or provide real-time tibiofemoral measurements.

One aspect of the present invention includes a knee arthrometer for measuring knee laxity comprising a femoral frame attachable to a leg of a test subject about a distal femur of the leg, the distal femur and the leg both having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion configured to contact the leg about the lateral epicondyle, the second arm portion configured to contact the leg about the medial epicondyle, the connecting portion connecting the first and second arm portions, the femoral motion sensor coupled to the femoral frame and configured to measure motion with six degrees of freedom and output femoral motion data associated with the measured motion; and a tibial frame attachable to the leg about a tibia, the tibia having a proximal end, a distal end, and an anterior crest, the tibial frame attachable to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom and output tibial motion data associated with the measured motion, wherein the femoral motion data and the tibial motion data are usable, in conjunction, to determine knee laxity.

The connecting portion may be curved to extend over the anterior side of the distal femur to accommodate the leg. The connecting portion may be sprung to bias the first arm portion toward the medial side of the leg and bias the second arm portion toward the lateral side of the leg such that the femoral frame is capable of being clamped to the leg. The femoral frame may be U-shaped, and the femoral securing mechanism may comprises a femoral securing strap, the femoral securing strap being attachable to the first arm portion and second arm portion and positionable about the posterior side of the leg. The femoral securing strap may be expandable, the femoral securing strap being configured to bias the first arm portion toward the second arm portion when attached to the first and second arm portions such that the femoral frame is capable of being clamped to the leg. The first and second arm portions may comprise an end opposite the connecting portion and a deformable contact pad coupled to the arm portion near the end, the deformable contact pad configured to contour to the leg. The tibial frame may further comprise a placement guide, the placement guide extending proximally and distally from the rigid frame portion and configured to guide a user in placing the tibial frame. The tibial securing mechanism may comprise a tibial securing strap, the tibial securing strap attachable to the rigid frame portion and positionable about the leg and tibia. The tibial securing strap may be expandable and configured to secure the tibial frame to the leg about the tibia. The tibial securing strap may comprise a hook and loop fastener and be configured to secure the tibial frame to the leg about the tibia. The connecting portion of the femoral frame may be positionable above the anterior side of the leg. The femoral motion sensor may be coupled to the connecting portion of the femoral frame. The tibial frame may be positionable over the anterior crest of the tibia.

Another aspect of the present invention includes a method for measuring knee laxity comprising: attaching a femoral frame to a leg of a test subject about a distal femur of the leg, the distal femur and the leg both having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion contacting the leg about the lateral epicondyle, the second arm portion contacting the leg about the medial epicondyle, the connecting portion connecting the first and second arm portions, the connecting portion positioned above the anterior side of the leg, the femoral motion sensor coupled to the femoral frame and configured to measure motion with six degrees of freedom; attaching a tibial frame to the leg about a tibia, the tibia having a proximal end and a distal end, the tibial frame attached to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom, the tibial securing mechanism attached to the rigid frame portion and positioned about the leg and tibia; moving the tibia relative to the femur in at least one cycle including knee flexion and extension; recording the motion of the femoral motion sensor and recording the motion of the tibial motion sensor through the at least one cycle; and determining knee laxity based on the recorded motion of the femoral and tibial motion sensors. The determining of the knee laxity may be based on calculations defined by a Cartesian coordinate system.

This method may further include moving the tibia in a first stage including a set of cycles; recording the motion of the femoral and tibial motion sensors during the first stage; and determining, based on the recorded motion during the first stage, a knee axis of rotation. The first stage may comprise three cycles, wherein for each cycle the tibia may be moved relative to the femur from an angle of fifteen degrees to an angle of sixty degrees and back to the angle of fifteen degrees.

This method may yet further include moving the tibia such that the knee may be at terminal extension during a knee laxity test in a second stage including a set of cycles, and holding the tibia such that the knee is at the terminal extension for a set period of time; recording the motion of the femoral and tibial motion sensors during the second stage; and determining, based on the recorded motion during the second stage and the knee axis of rotation, a first orthogonal coordinate system relative to the femur and a second orthogonal coordinate system relative to the tibia. The first orthogonal coordinate system may comprise a first axis along the knee axis of rotation, a second axis extending in an anterior direction, and a third axis extending in a proximal direction, and wherein the second orthogonal coordinate system may be coincident with the first orthogonal coordinate system when the knee is at terminal extension.

This method may yet further still include moving the tibia through a range of motion including flexion and extension from terminal extension to an angle of approximately seventy degrees relative to the femur, internal and external rotation, varus and valgus rotation, and anterior and posterior rotation in a third stage including a set of cycles; recording the motion of the femoral and tibial motion sensors during the third stage; determining cardan angle values between the first and second orthogonal coordinate systems; and determining translation distances of the second orthogonal coordinate system relative to the first orthogonal coordinate system. The determining of the knee laxity may include determining, based on the third set of cycles and corresponding recorded motion, a dynamic flexion and extension angle, an internal and external rotation value, and a varus and valgus rotation value, and an anterior and posterior rotation value.

Yet another aspect of the present invention includes a knee arthrometer for measuring knee tibiofemoral kinematics comprising: a femoral frame attachable to a leg of a test subject about a distal femur of the leg, the distal femur and the leg both having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion configured to contact the leg about the lateral epicondyle, the second arm portion configured to contact the leg about the medial epicondyle, the connecting portion connecting the first and second arm portions, the femoral motion sensor coupled to the femoral frame and configured to measure motion with six degrees of freedom and output femoral motion data associated with the measured motion; and a tibial frame attachable to the leg about a tibia, the tibia having a proximal end, a distal end, and an anterior crest, the tibial frame attachable to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom and output tibial motion data associated with the measured motion, wherein the femoral motion data and the tibial motion data are usable, in conjunction, to determine tibiofemoral kinematics in real-time.

The connecting portion may be curved to extend over the anterior side of the distal femur to accommodate the leg. The connecting portion may be sprung to bias the first arm portion toward the medial side of the leg and bias the second arm portion toward the lateral side of the leg such that the femoral frame is capable of being clamped to the leg. The femoral frame may be U-shaped. The femoral securing mechanism may comprise a femoral securing strap, the femoral securing strap attachable to the first arm portion and second arm portion and positionable about the posterior side of the leg. The femoral securing strap may be expandable, the femoral securing strap being configured to bias the first arm portion toward the second arm portion when attached to the first and second arm portions such that the femoral frame is capable of being clamped to the leg. The first and second arm portions may comprise an end opposite the connecting portion and a deformable contact pad coupled to the arm portion near the end, the deformable contact pad configured to contour to the leg. The contact pad may comprise a plurality of posts configured to contour to the leg. The plurality of posts of the contact pad may comprise a material configured to grip to the leg. The tibial frame may further comprise a placement guide, the placement guide extending proximally and distally from the rigid frame portion and configured to guide a user in placing the tibial frame. The tibial securing mechanism may comprise a tibial securing strap, the tibial securing strap being attachable to the rigid frame portion and positionable about the leg and tibia. The tibial securing strap may be expandable and configured to secure the tibial frame to the leg about the tibia. Therein the tibial securing strap may comprise a hook and loop fastener and may be configured to secure the tibial frame to the leg about the tibia. The connecting portion of the femoral frame may be positionable above the anterior side of the leg. The femoral motion sensor may be coupled to the connecting portion of the femoral frame. The tibial frame may be positionable over the anterior crest of the tibia. The femoral frame further may comprise femoral arm portions. The femoral frame may further comprise wire retention members. The femoral frame may further comprise being configured to secure the femoral sensor. The tibial frame may further comprise posts configured to secure the tibial sensor. The tibial frame may further comprise an upper femoral securing mechanism.

Yet another aspect of the present invention includes a method for measuring tibiofemoral kinematics comprising: attaching a femoral frame to a leg of a test subject about a distal femur of the leg, the distal femur and the leg both having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion contacting the leg about the lateral epicondyle, the second arm portion contacting the leg about the medial epicondyle, the connecting portion connecting the first and second arm portions, the connecting portion positioned above the anterior side of the leg, the femoral motion sensor coupled to the femoral frame and configured to measure motion with six degrees of freedom; attaching a tibial frame to the leg about a tibia, the tibia having a proximal end and a distal end, the tibial frame attached to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom, the tibial securing mechanism attached to the rigid frame portion and positioned about the leg and tibia; moving the tibia relative to the femur in at least one cycle including at least one of knee flexion and extension, internal-external knee movement, and adduction-abduction knee movement; recording the motion of the femoral motion sensor and recording the motion of the tibial motion sensor through the at least one cycle; and determining tibiofemoral kinematics in real-time based on the recorded motion of the femoral and tibial motion sensors. This method may further comprise: moving the tibia in a first stage including a set of cycles; recording the motion of the femoral and tibial motion sensors during the first stage; and determining, based on the recorded motion during the first stage, a knee axis of rotation.

The first stage may comprise three cycles, wherein for each cycle the tibia may be moved relative to the femur from a first angle to a second angle different than the first angle. The tibia may comprise each tibia of the test subject and the knee may comprise each knee of the test subject, the method further comprising: moving the tibias such that the knees are brought together; recording the motion of the femoral and tibial motion sensors; and determining, based on the recorded motion, a Cartesian coordinate system. The tibia may comprise each tibia of the test subject and the knee may comprise each knee of the test subject, the method further comprising: moving the tibias such that the knees are outward from one another; recording the motion of the femoral and tibial motion sensors; and determining, based on the recorded motion, a Cartesian coordinate system. The tibia may comprise each tibia of the test subject and the knee comprises each knee of the test subject, the method further comprising: moving the tibias such that the knees are neutral to one another; recording the motion of the femoral and tibial motion sensors; and determining, based on the recorded motion, a Cartesian coordinate system. The Cartesian coordinate system may define a search space for calculating a knee angle of rotation.

The determining of the tibiofemoral kinematics in real-time may include determining tibiofemoral kinematics resulting from measurements taken during a step-up test. The determining the tibiofemoral kinematics in real-time may include determining tibiofemoral kinematics resulting from measurements taken during performance of a squatting test. The squatting test may comprise the test subject performing a neutral dual limb squatting task, a Valgus squatting task, and/or a Varus squatting task.

Yet another aspect of the present invention includes a method for measuring tibiofemoral kinematics comprising: attaching a femoral frame to a leg of a test subject about a distal femur of the leg, the distal femur and the leg both having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion contacting the leg about the lateral epicondyle, the second arm portion contacting the leg about the medial epicondyle, the connecting portion connecting the first and second arm portions, the connecting portion positioned above the anterior side of the leg, the femoral motion sensor coupled to the femoral frame and configured to measure motion with six degrees of freedom; attaching a tibial frame to the leg about a tibia, the tibia having a proximal end and a distal end, the tibial frame attached to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom, the tibial securing mechanism attached to the rigid frame portion and positioned about the leg and tibia; moving the tibia relative to the femur in at least one cycle of a functional task; recording the motion of the femoral motion sensor and recording the motion of the tibial motion sensor through the at least one cycle; and determining tibiofemoral kinematics based on the recorded motion of the femoral and tibial motion sensors.

The determining of the tibiofemoral kinematics may be based on calculations defined by a Cartesian coordinate system. The determining of the tibiofemoral kinematics may be based on calculations defined by an orthogonal coordinate system.

These are merely some of the innumerable aspects of the present invention and should not be deemed an all-inclusive listing of the innumerable aspects associated with the present invention. These and other aspects will become apparent to those skilled in the art in light of the following disclosure and accompanying drawings. The description and specific examples in this summary are intended for purposes of illustration only and are not intended to limit the scope of the present disclosure.

### BRIEF DESCRIPTION OF THE DRAWINGS

The accompanying drawings, which are incorporated in and form a part of the specification, illustrate the embodiments of the present disclosure and together with the description, serve to explain the principles of the disclosure.

FIG. 1 illustrates a schematic view of a knee arthrometer relative to bones within the leg according to a first embodiment of the present invention.

FIG. 2 illustrates a perspective view of the knee arthrometer shown in FIG. 1 relative to a leg.

FIGS. 3A-3G illustrate a flow chart for a first stage of a method of determining movement of the knee using the knee arthrometer shown in FIG. 1.

FIGS. 4A and 4B illustrate a flow chart for a second stage of a method of determining movement of the knee using the knee arthrometer shown in FIG. 1.

FIG. 5 illustrates a flow chart for a third stage of a method of determining movement of the knee using the knee arthrometer shown in FIG. 1.

FIGS. 6A and 6B illustrate a modified knee arthrometer.

FIGS. 7A-7E illustrate aspects of a femoral frame of the knee arthrometer shown in FIGS. 6A and 6B.

FIGS. 8A-8C illustrate aspects of a tibial frame of the knee arthrometer shown in FIGS. 6A and 6B.

FIG. 9 illustrates a CT scan of the knee arthrometer shown in FIGS. 6-8, secured to a leg of a test subject.

FIG. 10 illustrates a step-up task performed by a test subject wearing the knee arthrometer as shown in FIGS. 6-8.

FIGS. 11A-11C illustrate various squatting tasks performed by a test subject wearing the knee arthrometer as shown in FIGS. 6-8.

FIGS. 12A-12J illustrate a flow chart for a first, second and third stage of a method of determining movement of the knee using the knee arthrometer according to a second embodiment of the present invention, where FIGS. 12A-12G illustrate the first stage (Stage 1), FIGS. 12H and 12J illustrate the second stage (Stage 2), and FIG. 12J illustrates the third stage (Stage 3).

FIG. 13 illustrates test results for various principal components with respect to flexion, internal-external, and adduction-abduction knee movements.

FIG. 14 illustrates test results for various principal components for various squatting tasks performed with internal-external and adduction-abduction knee movements.

FIGS. 15A-15I illustrate test results for various principal components and flexion, internal-external and adduction-abduction knee movements.

FIGS. 16A-16C illustrate test results for a neutral squatting task with respect to flexion, internal-external and adduction-abduction knee movements.

FIGS. 17A-17C illustrate test results for a Valgus squatting task with respect to flexion, internal-external and adduction-abduction knee movements.

FIGS. 18A-18C illustrate test results for a Varus squatting task with respect to test subject averages for flexion, internal-external and adduction-abduction knee movements.

FIG. 19 illustrates sensor placement for assessment of an abnormal muscle reaction (e.g., muscle spasticity) according to one aspect of the present invention.

FIG. 20 illustrates a plot of displacement test data obtained from a muscle spasticity test conducted using sensor placement in accordance with that shown in FIG. 19.

FIG. 21 illustrates a plot of joint angle and velocity test data obtained from a muscle spasticity test conducted using sensor placement in accordance with that shown in FIG. 19.

FIG. 22 illustrates a step-up task performed by a test subject wearing a knee arthrometer according to an embodiment of the invention and an ankle foot orthotic.

FIGS. 23A, 23B and 23C illustrate step-up test results with respect to flexion, internal-external, and adduction-abduction knee movements when a test subject is wearing an ankle foot orthotic.

FIG. 24 illustrates step-up cycle data obtained from an ankle foot orthotic test in accordance with that shown in FIGS. 22, 23A, 23B and 23C.

FIG. 25 illustrates extent of motion data obtained from an ankle foot orthotic test in accordance with that shown in FIGS. 22, 23A, 23B and 23C.

FIG. 26 illustrates a methodology for performing techniques according to the present invention.

Reference characters in the written specification indicate corresponding items shown throughout the drawing figures.

### DETAILED DESCRIPTION

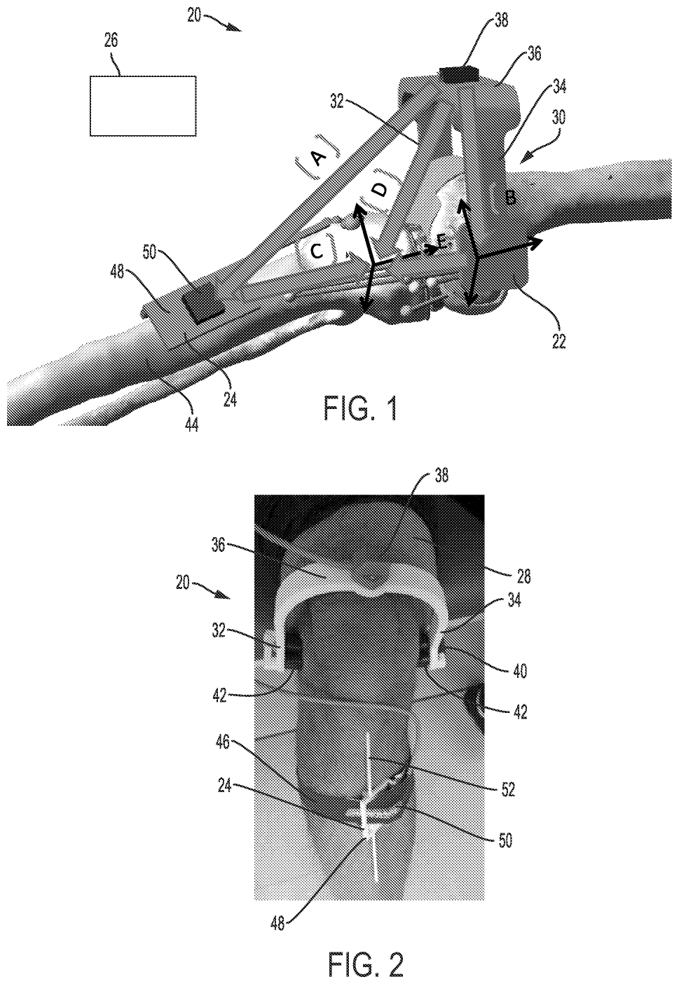

Referring to FIGS. 1 and 2, a knee arthrometer 20 for measuring knee laxity is depicted according to a first embodiment. The knee arthrometer 20 may be 3D-printed (e.g., using nylon), and includes a femoral frame 22 and a tibial frame 24. The femoral frame 22 may be referred to as a femoral clamp, and the tibial frame 24 may be referred to as a tibial clamp. The femoral frame 22 and the tibial frame 24 cooperate with a processor 26 to measure and determine movement of the tibia relative to the femur. As described in greater detail later herein, the processor 26 uses data from sensors on the femoral frame 22 and the tibial frame 24 to calculate joint laxity including the dynamic flexion/extension angle, internal/external rotation, anterior/posterior translation, and varus/valgus rotation of the tibia relative to the femur. The processor 26 may be part of a larger computer system (not shown) that is capable of processing data acquired from the sensors.

The femoral frame 22 is attachable to a leg 28 about a distal femur 30. The distal femur 30 and the leg 28 both have a medial side, a lateral side, an anterior side, a posterior side, a lateral epicondyle, and a medial epicondyle. The femoral frame 22 includes a first arm portion 32, a second arm portion 34, a connecting portion 36, a femoral motion sensor 38, and a femoral securing mechanism 40. The first arm portion 32 is configured to contact the leg 28 about the lateral epicondyle. The second arm portion 34 is configured to contact the leg 28 about the medial epicondyle. The connecting portion 36 connects the first 32 and second 34 arm portions. The femoral motion sensor 38 is coupled to the femoral frame 22 and is configured to measure motion with six degrees of freedom and output femoral motion data associated with the measured motion. Generally the femoral motion sensor 38 is positioned on the connecting portion 36, but it can alternatively be positioned elsewhere on the femoral frame 22. The femoral securing mechanism 40 secures the femoral frame 22 to the leg 28.

The connecting portion 36 is curved to extend over the anterior side of the distal femur 30 to accommodate the leg 28. The connecting portion 36 may be sprung to bias the first arm portion 32 toward the medial side of the leg and bias the second arm portion 34 toward the lateral side of the leg such that the femoral frame 22 is capable of being clamped to the leg 28. This configuration secures the femoral frame 22 to the leg 28. For example, and without limitation, the femoral frame 22 may be U-shaped.

The femoral securing mechanism 40 is a femoral securing strap (e.g., as shown in FIG. 2). The femoral securing strap is attachable to the first arm portion 32 and second arm portion 34 and is positionable about the posterior side of the leg 28. The femoral securing strap may be configured to be expandable. The femoral securing strap is configured to bias the first arm portion 32 toward the second arm portion 34 when attached to the first and second arm portions 32, 34 such that the femoral frame 22 is capable of being clamped to the leg 28.

The first and second arm portions 32, 34 may each include an end opposite the connecting portion 36 and a deformable contact pad 42 coupled to the arm portion near the end. The deformable contact pad 42 is configured to contour to the leg 28. The deformable contact pad 42 may improve patient comfort and/or facilitate securing of the femoral frame 22 to the leg 28.

The tibial frame 24 is attachable to the leg 28 about a tibia 44. The tibia has a proximal end, a distal end, and an anterior crest. The tibial frame 24 attaches to the leg 28 about the tibia 44 closer to the proximal end of the tibia 44 than the distal end of the tibia. The tibial frame includes a tibial securing mechanism 46, a rigid frame portion 48, and a tibial motion sensor 50. The tibial motion sensor 50 is coupled to the rigid frame portion 48 and is configured to measure motion with six degrees of freedom and output tibial motion data associated with the measured motion. The femoral motion data and the tibial motion data are usable, in conjunction and by a processor, to determine knee laxity as described in greater detail later herein. The femoral motion sensor 38 and/or the tibial motion sensor 50 may communicate with the processor using a wired or wireless data and/or power connection.

The tibial frame 24 may further comprise a placement guide 52. The placement guide 52 extends proximally and distally from the rigid frame portion 48 and is configured to guide a user in placing the tibial frame 24. For example, and without limitation, the placement guide 52 is a protrusion, rod, or the like, having an end opposite the rigid frame portion 48. The user may place the tibial frame by centering the end of the placement guide 52 relative to the knee (e.g., centered on the patella or more specifically the tibial tuberosity).

The tibial securing mechanism 46 may be configured as a tibial securing strap. The tibial securing strap is attachable to the rigid frame portion 48 and is positionable about the leg 28 and tibia 44. The tibial securing strap may be expandable and is configured to secure the tibial frame 24 to the leg 28 about the tibia 44. For example, and without limitation, the tibial securing strap may be a hook and loop fastener and stretchable material such that the tibial frame 24 is securable to the leg 28 about the tibia 44. The tibial frame 24 may be configured to be positionable over the anterior crest of the tibia 44.

In operation, an electromagnetic motion tracker measures the position and orientation of the tibial motion sensor 50 relative to the femoral motion sensor 38. For example, and without limitation, the electromagnetic motion tracking system, tibial motion sensor, and femoral motion sensor may be of the type used in the Polhemus™ Patriot™ system, and in particular the Polhemus™ Patriot™ two sensor 6DOF (e.g., six degrees of freedom) electromagnetic tracker. The femoral motion sensor 38 is attached to the femoral frame 22 that is positioned on the distal femur. The tibial motion sensor 50 is attached to the tibial frame 24 that is placed on the shin (tibia). The femoral frame 22 is placed over the distal femur such that rubber covered pegs (e.g., contact pads 42) on the interior of the frame surround the medial and lateral epicondylar eminences. The opening of the femoral frame 22 is increased prior to placement on the femur and when released, pressure is applied to the femur by the femoral frame 22, holding it in place. A strap that goes under the femur helps secure the femoral frame 22. The patient is in a seated position when the femoral frame 22 is positioned by the operator such that the femoral motion sensor 38 is above the epicondylar axis of the femur. Different sized femoral frames 22 accommodate different sized knees.

The tibial frame 24 is placed over the anterior crest of the tibia 44 such that the tibial motion sensor 50 is located on the flat surface of the tibia just medial to the crest. A guide (e.g., placement guide 52) is used to assist the operator in placing the tibial frame 24. The tip of the guide is placed on the tibial tuberosity such that the distal distance of the tibial frame 24 from the knee is consistent. A strap (e.g., tibial securing mechanism 46) wraps around the calf and secures the tibial frame 24 to the tibia 44.

During measurements, outputs of the electromagnetic motion tracker are the six degree-of-freedom translations and orientations of the tibial motion sensor 50 relative to the femoral motion sensor 38. The preferred orientation output of the motion tracker is in the form of Euler Parameters (quaternions). The measured orientation data is converted into a rotation matrix designated as “A” in FIG. 1. After placement of the tibial frame 24 and femoral frame 22, a physical therapist, surgeon, athletic trainer, or other user/operator passively moves the tibia 44 relative to the femur 30. During these movements, only the tibia 44 is moved as the patient remains seated on a plinth or table.

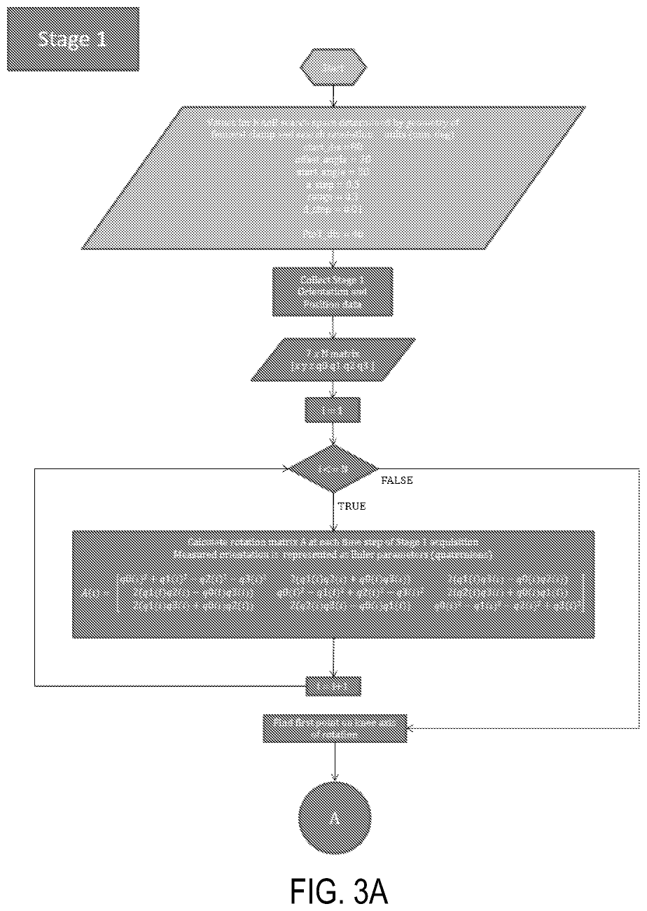

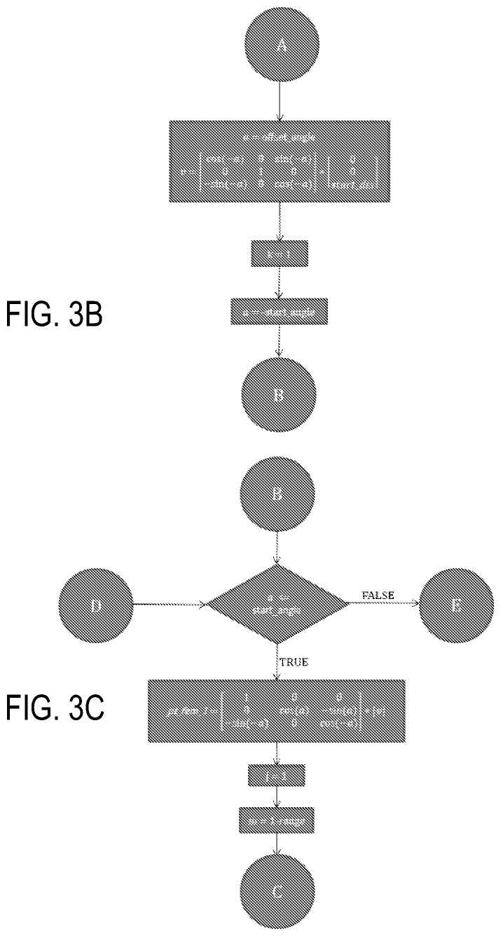

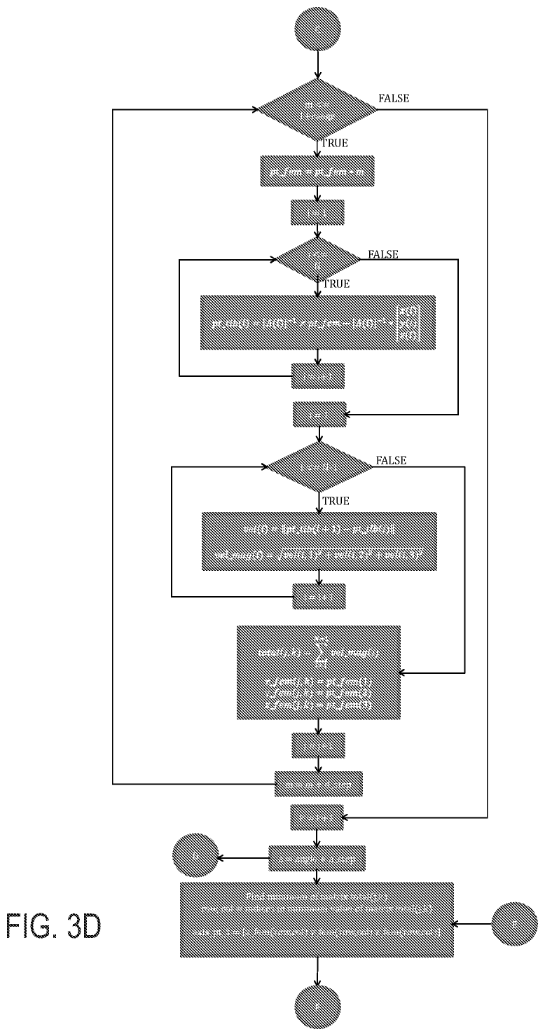

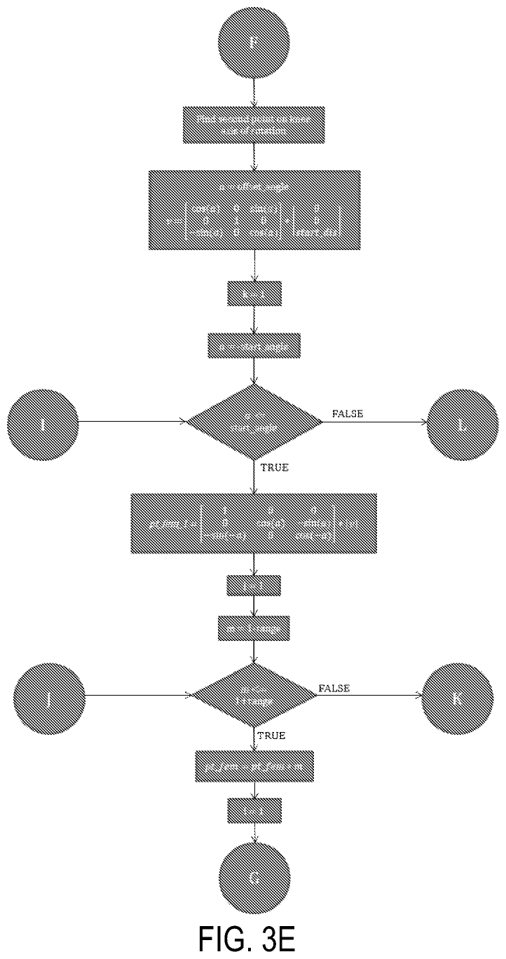

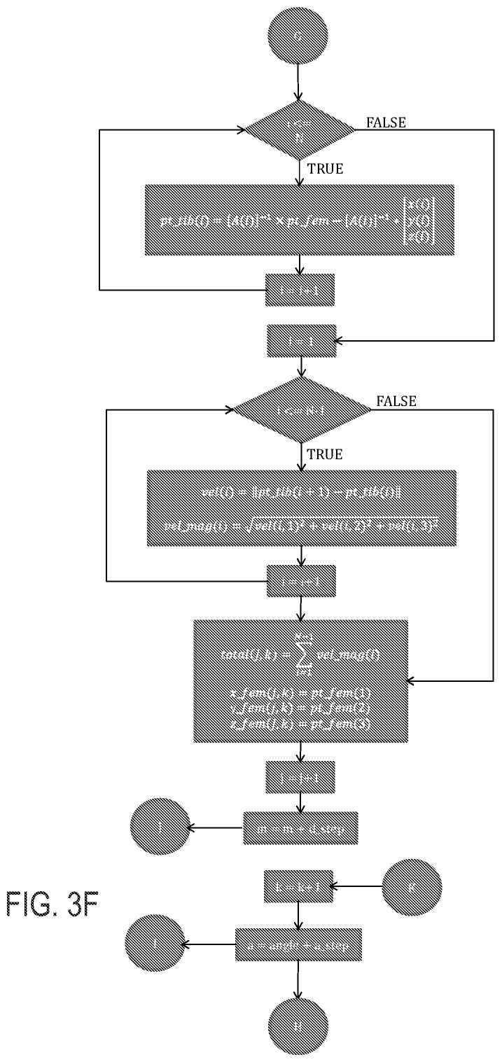

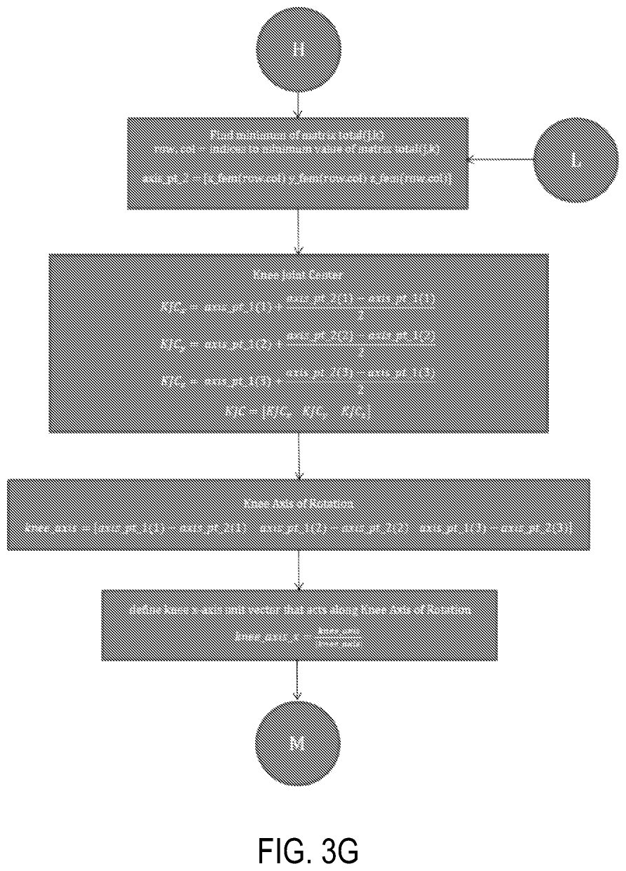

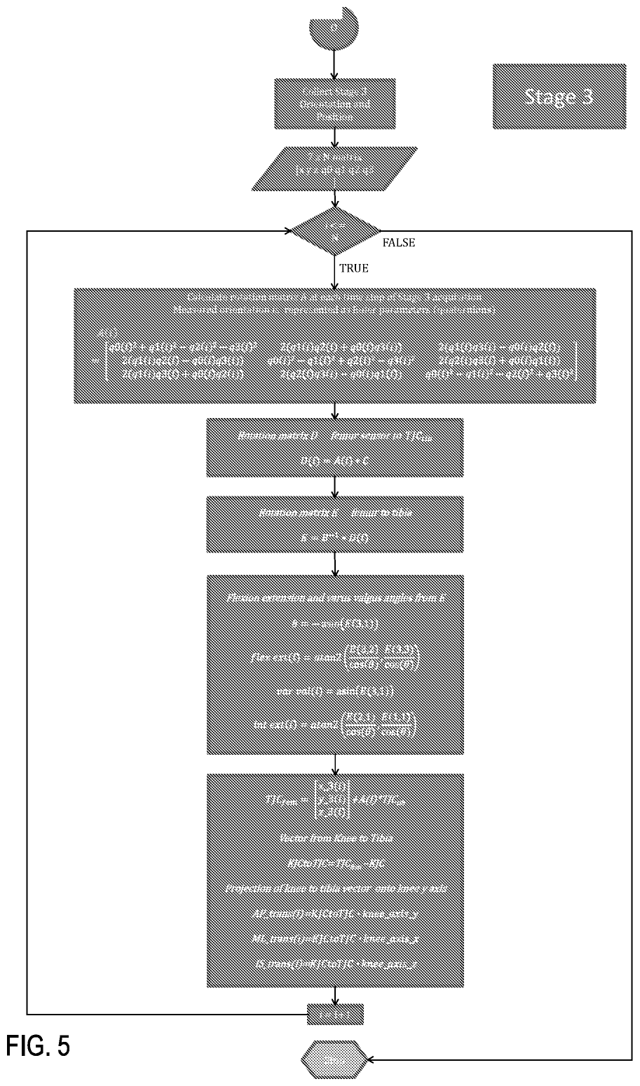

Referring now to FIGS. 3-5, the passive motion measurements are done in three stages (Stage 1, Stage 2 and Stage 3) using an algorithm.

In Stage 1 (FIGS. 3A-3G), the tibia 44 is passively moved such that the knee flexion cycles from approximately 15 degrees to 60 degrees then back to 15 degrees, for example. This flexion/extension cycle is repeated for a total of 3 cycles, for example. However, alternative movements where the knee flexes a greater or lesser amount and/or the number of cycles is greater or lesser are likewise envisioned and within the scope of the present invention (although it is generally preferable to not extend more than 15 degrees (e.g., terminal extension) to reduce the impact of a knee screw-home mechanism which can influence determination of a Knee Axis of Rotation (KAoR)). Also, Stage 1 flexion/extension motion does not need to be passive. A test subject patient can sit on the edge of a plinth and cycle their leg back and forth. The output of Stage 1 is a KAoR in the femoral orthogonal coordinate system. The calculations to determine the KAoR are detailed in the flow charts illustrated in FIGS. 3A-3G.

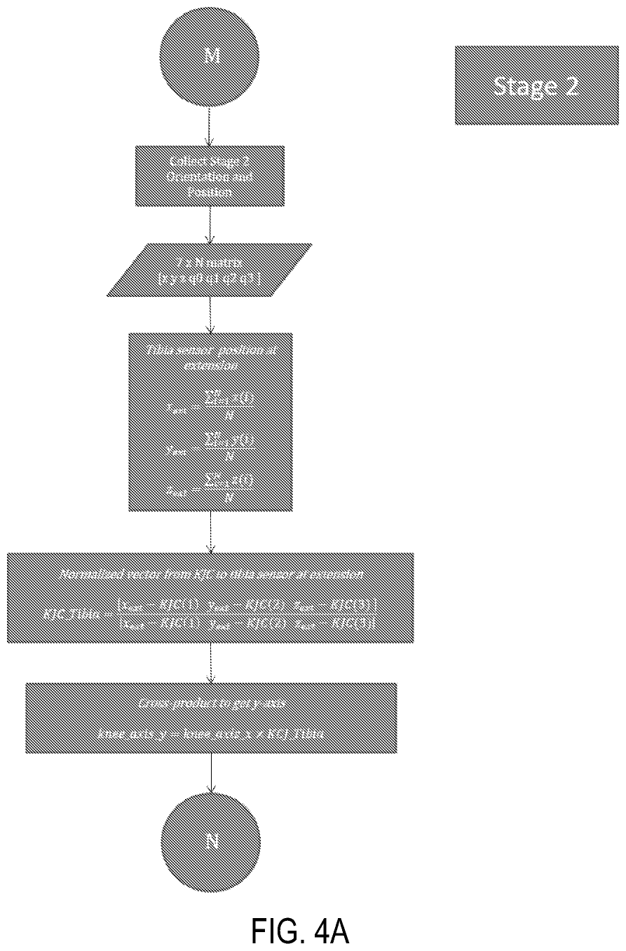

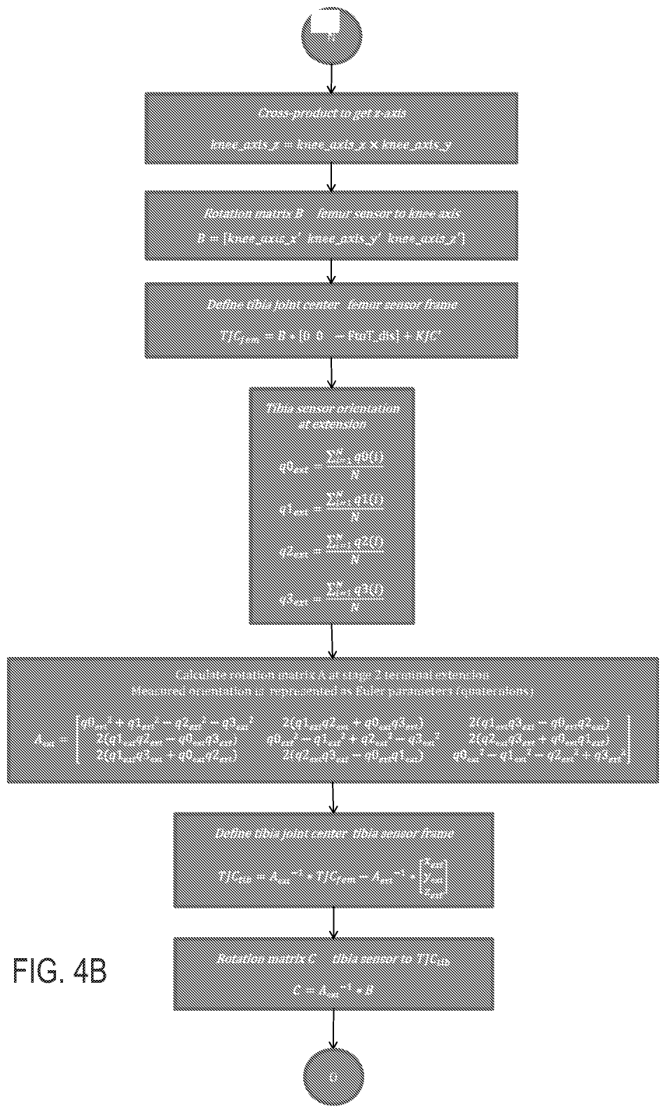

In Stage 2 (FIGS. 4A and 4B), the tibia 44 is moved such that the knee is at terminal extension. This position is held for 2 seconds, although alternative durations where the position is held for other amounts of time are envisioned. The output of Stage 2 is an orthogonal coordinate system relative to the femur 30 positioned in the femur 30 where x acts along the KAoR and points in the lateral direction, the y-axis points in the anterior direction, and the z-axis points proximally (e.g., as illustrated in FIG. 1). The output of Stage 2 also includes an orthogonal coordinate system relative to the tibia 44 positioned in the tibia 44 with orientation coincident with terminal extension (e.g., as illustrated in FIG. 1). The calculations to determine the orthogonal coordinate systems are detailed in the flow charts illustrated in FIGS. 4A and 4B.

In Stage 3, the tibia 44 is moved through its dynamic passive range of motion. This may include flexion-extension from terminal extension to˜70 degrees, internal external-rotation, varus-valgus rotation, and anterior-posterior translation. The output of Stage 3 is Cardan angles and translation of the tibia 44 (e.g., the tibial orthogonal coordinate system) relative to the femur 30 (e.g., the femoral orthogonal coordinate system). One or more of the outputs are in coordinates relative to/in the femoral orthogonal coordinate system.

With reference general to FIGS. 3-5, the measured motion of the tibial motion sensor 50 relative to the femoral motion sensor 38 (“A”, during Stage 1 movement) provides input to the algorithm that determines the knee axis of rotation (KAoR), relative to the femoral motion sensor 38. The algorithm is a functional joint axis approach that defines a point on the femur 30 in a femoral orthogonal coordinate system and in a tibial motion sensor 50 frame for each position and orientation acquisition of Stage 1. The velocity magnitude, over the Stage 1 acquisition, of the vector locating the point in the tibial motion sensor 50 frame will be minimal at a point that lies along the anatomical KAoR. Two points along the KAoR are identified. A point midway between the medial and lateral epicondylar eminences of the knee and along the KAoR defines the origin of the femoral orthogonal coordinate system. The x-axis of the femoral orthogonal coordinate system is defined to act along the KAoR and point laterally.

Stage 2 moves the knee into terminal extension (e.g., for the laxity test described above, when the subject is sitting on a plinth, a therapist, for example, would move the leg to terminal extension). Motion tracker output of the femoral motion sensor 38 and tibial motion sensor 50 at this static position is used to define the remaining axes of the femoral orthogonal coordinate system. A vector is created from an origin of the femoral orthogonal coordinate system to an origin of the tibial motion sensor 50. The cross product between the femoral orthogonal coordinate system x-axis and the vector between the femoral orthogonal coordinate system and the tibial motion sensor 50 frame origin defines the y-axis of the femoral orthogonal coordinate system. The y-axis points anteriorly from the femur. The cross-product between the femoral orthogonal coordinate system x-axis and y-axis defines femoral orthogonal coordinate system z-axis. The z-axis approximately acts along the long axis of the femur and points in the proximal direction. A rotation matrix from the femoral motion sensor 38 to the femoral orthogonal coordinate system is then defined. This rotation matrix, along with the origin of the femoral orthogonal coordinate system in femoral motion sensor 38 coordinates, defines the location and orientation of the femoral orthogonal coordinate system relative to the femoral motion sensor 38. This is designated “B” in FIG. 1. As the femoral orthogonal coordinate system is defined on the same rigid body as the femoral motion sensor 38, B is a constant.

In the Stage 2 terminal extension position, a tibial orthogonal coordinate system is defined. The tibial orthogonal coordinate system is coincident with the femoral coordinate system with its origin translated distally a set distance from the femoral orthogonal coordinate system along the z-axis of the femoral coordinate system. The origin location and orientation of the tibial orthogonal coordinate system relative to the tibial motion sensor 50 is then determined. This is designated “C” in FIG. 1. C is a constant. Motion of the tibial orthogonal coordinate system relative to the femoral motion sensor 38 (“D” in FIG. 1) is then determined as well as the motion of the tibial orthogonal coordinate system relative to the femoral orthogonal coordinate system (“E” in FIG. 1).

Motion of the tibial orthogonal coordinate system relative to the femoral coordinate system, E, during the passive motion of Stage 3 is used to calculate joint laxity including the dynamic flexion/extension angle, internal/external rotation, anterior/posterior translation, and varus/valgus rotation of the tibia 44 relative to the femur 30. The algorithm details and mathematical equations used in these determinations/calculations are illustrated in FIGS. 3-5. The algorithm shown in FIGS. 3-5 may be executed on a computer system such as the system including processor 26 as shown in FIG. 1, or a separate system (e.g., cloud-based). Alternatively, anatomical coordinates could be converted to polar coordinates.

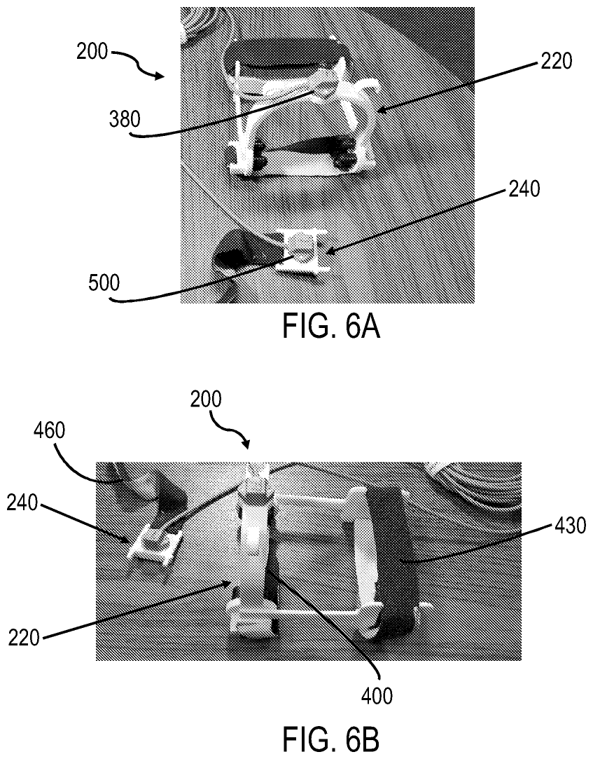

FIGS. 6-11 relate to a modified knee arthrometer of the present application. The modified knee arthrometer includes the same general features of the knee arthrometer 20 of the first embodiment as described above, except that the knee arthrometer includes certain structural features not shown as part of knee arthrometer 20.

As illustrated in FIGS. 6A and 6B, the modified knee arthrometer 200 is comprised of a femoral frame 220 and a tibial frame 240. The knee arthrometer 200 can be formed in various sizes to accommodate different knee sizes (e.g., widths). For example, various different sizes for the femoral frame 220 can be used to accommodate different-sized knees. For conducting tests, the femoral frame 220 is outfitted with a femoral motion sensor 380 and a femoral securing mechanism 400, and the tibial frame 240 is outfitted with a tibial securing mechanism 460 and a tibial motion sensor 500. The sensors 380 and 500 may be the same type as those above (e.g., sensors 38 and 50). As shown in FIGS. 6A and 6B, the sensors 380 and 500 may be secured to the femoral frame 220 and the tibial frame 240 by posts that are formed on the respective frames and extend through receiving holes in the respective sensors. These posts are described in more detail below. Additional securing means (e.g., adhesive, other straps, resistive bands, etc.) may also be used to secure the sensors to the frames.

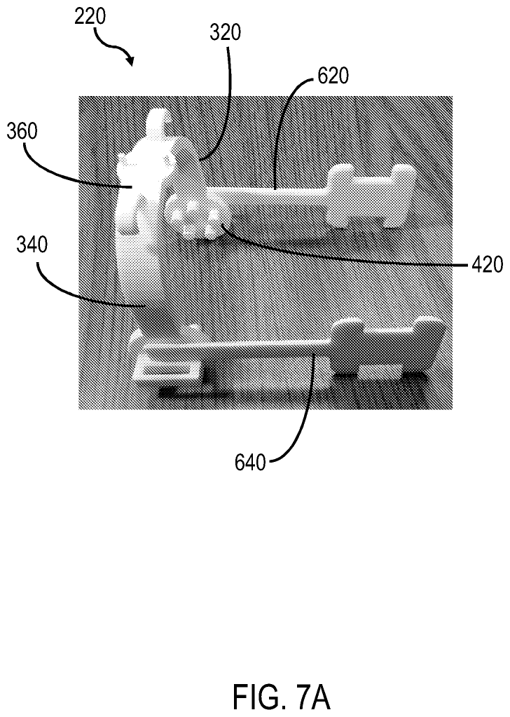

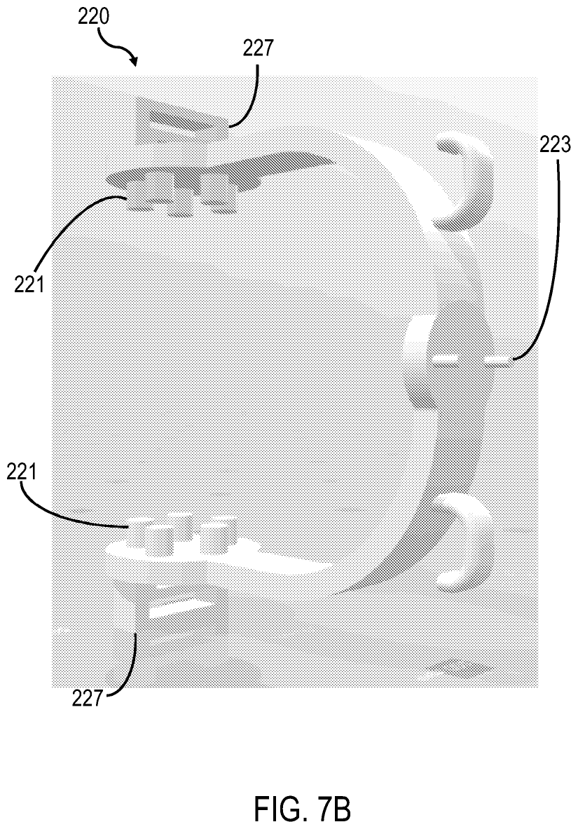

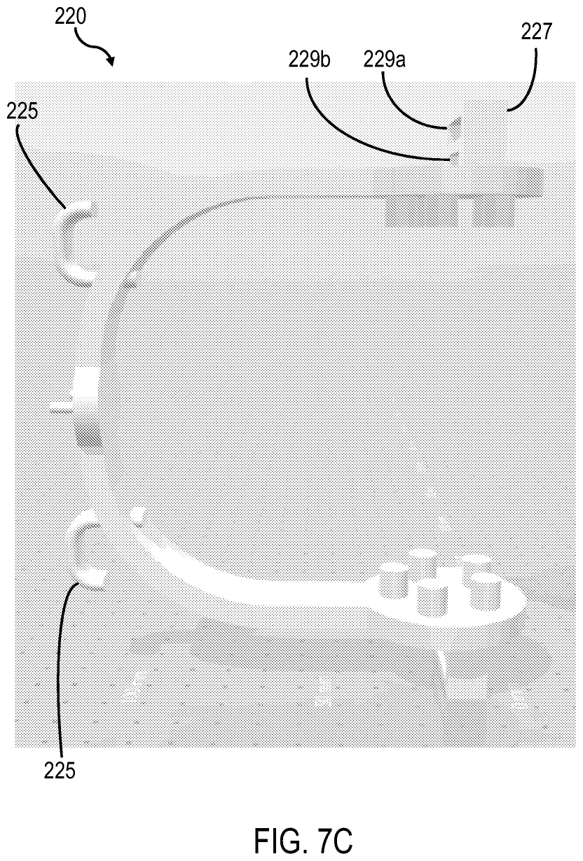

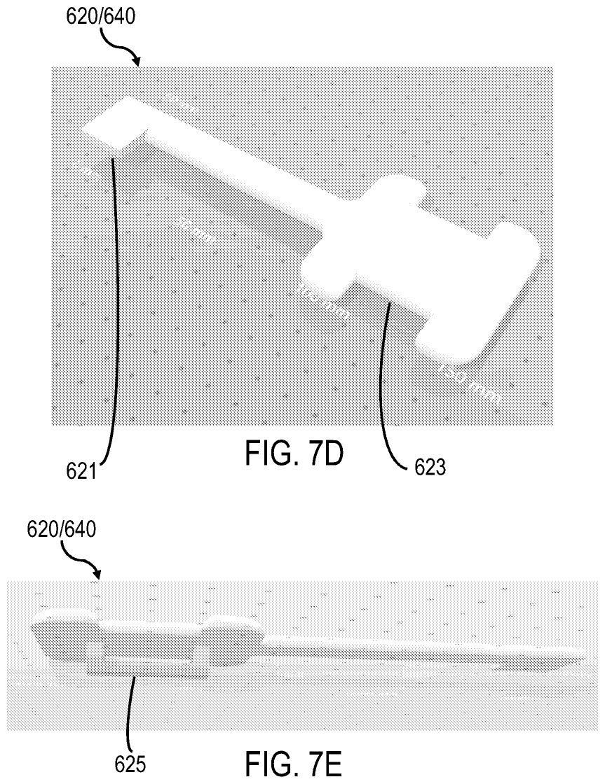

As illustrated in FIGS. 7A-7E, the femoral frame 220 includes a first arm portion 320, a second arm portion 340, and a connecting portion 360 therebetween. The femoral frame 220 can further include optional femoral arm portions 620 and 640 that correspond with the first and second arm portions 320 and 340, respectively. With reference to the femoral frame 220 shown in FIGS. 6A and 6B, the femoral arm portions 620 and 640 can receive an upper femoral securing mechanism 430 (such as a strap) for securing the femoral arm portions 620 and 640 to a femur portion of a leg of the test subject. The ends of the first and second arm portions 320 and 340 of the femoral frame 220 each include a contact pad 420 that comprises (e.g., five) posts 221, as illustrated in FIG. 7B. These posts 221 can be covered with rubber tips or similar material, and aid in compression of the frame onto the femur of the test subject. Due to the location of contact pads 420 at the ends of the first and second arm portions 320 and 340 of the femoral frame 220, five posts contact the medial femur portion of the test subject, and five posts contact the lateral femur portion of the test subject. More or less than five posts can be used, and spring-loading techniques and/or mechanisms may be used in conjunction with the contact pads 420 and/or the posts 221 to further increase compression and a secure fit. FIG. 7B also shows fastening mechanisms 227 at each end of the femoral frame 220 that aid in the fastening of the femoral frame 220 to a leg of a test subject, as described below in more detail. FIG. 7B further illustrates optional sensor posts 223 (e.g., a pair) that secure the femoral motion sensor 380 to the center of the connecting portion 360, as shown in FIGS. 6A and 6B. FIG. 7C illustrates additional features of the femoral frame 220, including (i) optional retention members 225 and (ii) the fastening mechanisms 227 that are used in conjunction with the femoral securing mechanism 400 to enable a secure fit on the leg of the test subject (see, e.g., FIG. 6B). For example, the retention members 225 may be used in combination with other restraints (e.g., other straps, resistive bands, etc.) to further restrain the sensor on the frame, and/or can be used to guide/retain wires connected to the various sensors shown in FIGS. 6A and 6B. The fastening mechanisms 227 can include a first slot 229a for receiving the femoral securing mechanism 400 and a second slot 229b for interfacing with the femoral arm portion(s) 620/640. FIG. 7D illustrates an example of the femoral arm portion(s) 620/640. One end of the femoral arm portion(s) 620/640 includes a tip portion 621 for insertion into slot 229b, and the other end includes indents 623 for receiving upper femoral securing mechanism 430, as shown in FIGS. 6A, 6B and/or 7A. As illustrated by FIG. 7E, the femoral arm portion(s) 620/640 can also be formed with a fastening mechanism 625 to secure the upper femoral securing mechanism 430 to further enable a secure fit to a leg of the test subject.

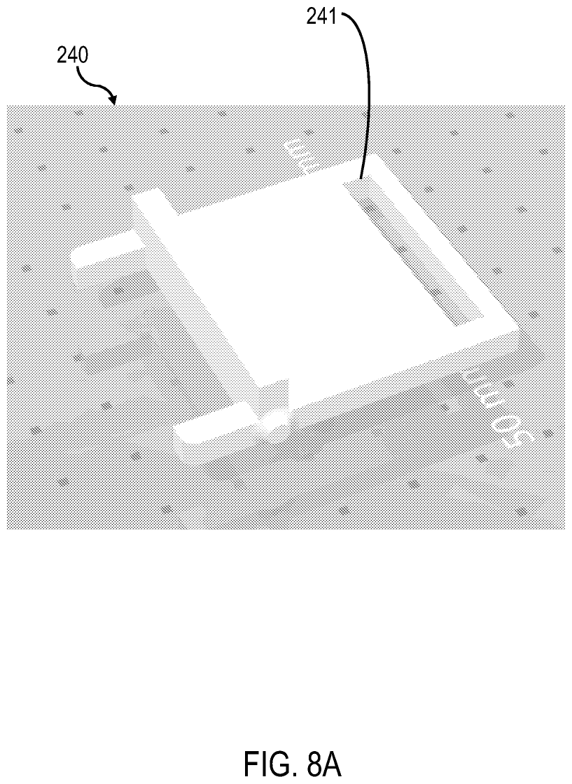

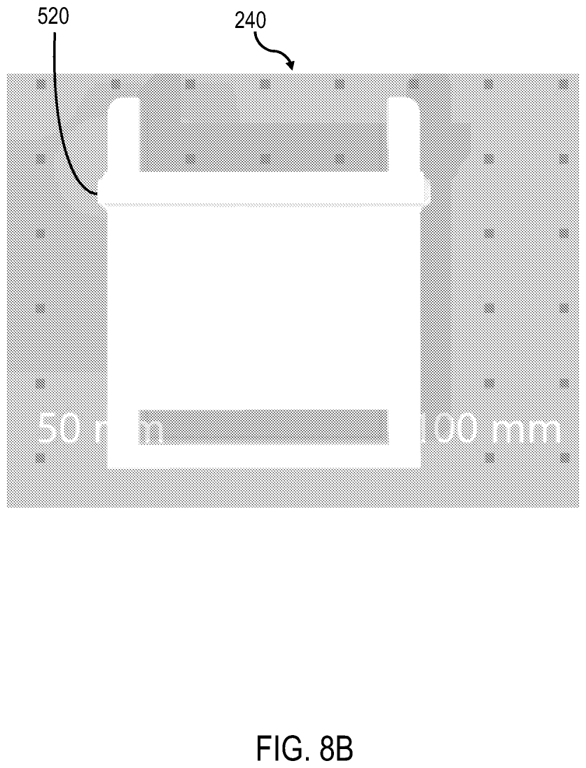

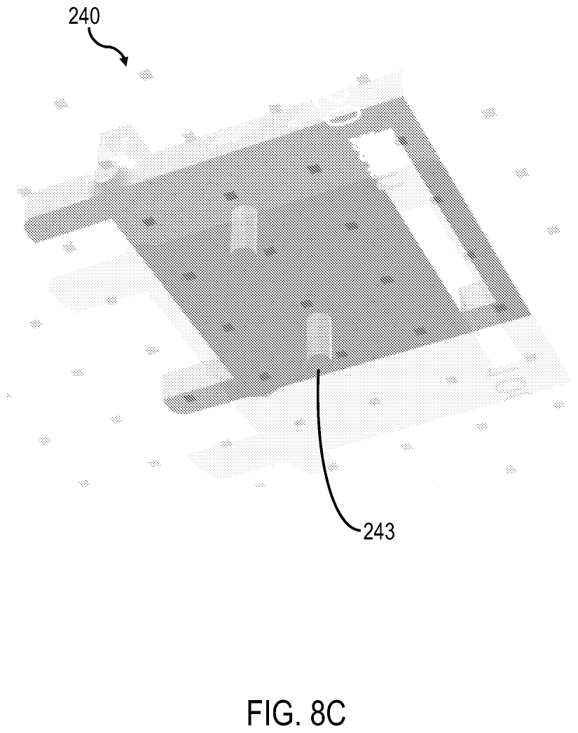

FIGS. 8A-8C illustrate tibial frame 240, which includes a slot 241 for securing the tibial securing mechanism 460 to enable a tight fit on the leg of the test subject, as shown in FIGS. 6A and 6B. FIG. 8B illustrates placement guides 520 that may be formed on each end of the tibial frame 240. These placement guides may be formed to a certain desired length, as shown by like element 52 in FIG. 2. As illustrated in FIG. 8C, the tibial frame 240 can include optional sensor posts 243 (e.g., a pair) for securing the tibial motion sensor 500, as shown in FIGS. 6A and 6B.

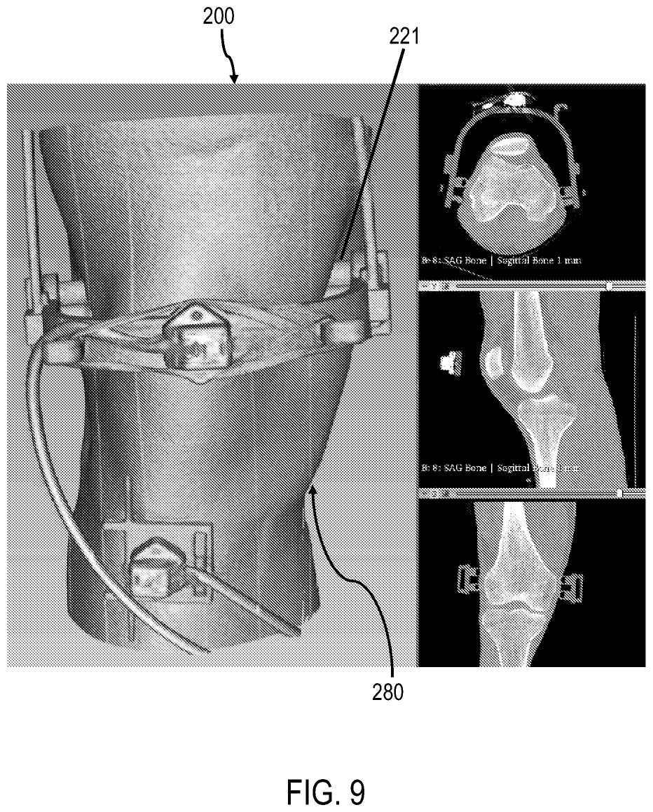

FIG. 9 illustrates a CT scan of the knee arthrometer 200 (which includes, for example, the various frames (e.g., 220, 240, see FIGS. 6A, 6B), sensors (e.g., 380, 500, see FIG. 6A) and securing mechanisms (e.g., 400, 430, 460, see FIG. 6B) as described above) secured to the leg 280 of the test subject. For example, as shown in FIG. 9, the posts 221 provide secure clamping of the knee arthrometer 200 to the leg 280 of the test subject.

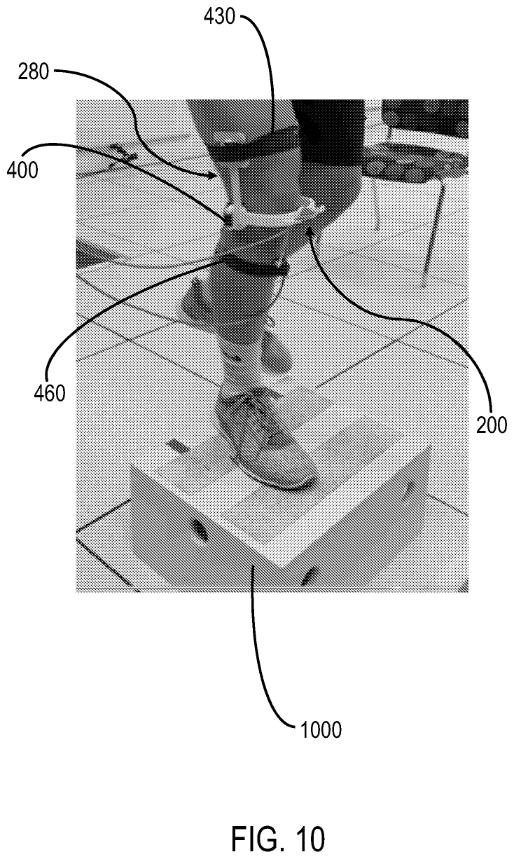

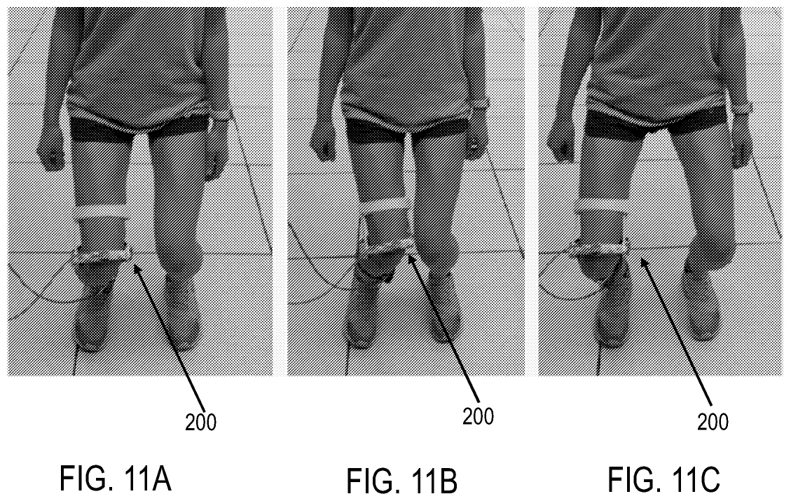

FIGS. 10 and 11A-11C illustrate various tasks that the test subject may perform while wearing the knee arthrometer 200. As illustrated in FIG. 10, while wearing the knee arthrometer 200, the test subject performs a stepping (e.g., step-up) task using a box 1000 of a certain height. FIG. 10 also illustrates how the various securing mechanisms (e.g., 400, 430, 460, see FIG. 6B) secure to the leg 280 of a test subject, and how the (wire) retention member(s) 225 can direct wires that are connected to the various sensors safely to the side during testing. FIGS. 11A-11C illustrate a test subject performing various squatting tasks while wearing the knee arthrometer 200. FIG. 11A illustrates a neutral squat task, FIG. 11B illustrates a Valgus squat task, and FIG. 11C illustrates a Varus squat task, described in more detail below. FIGS. 10 and 11 represent use of the present KATS for testing two types of functional tasks. However, the present KATS may be used for testing of other (dynamic) functional tasks including treadmill walking, lateral step-down, lunging, hopping, etc., all such movements being included within the scope of the present invention. For example, for functional tests, a test subject may be asked to stand on one leg while actively flexing and/or extending the knee (e.g., the flexion extension motion in Stage 1 (as described above and below) can be used to determine the KAoR). The flexing may include the test subject standing with their knee in full extension.

With respect to the measurements and calculation of KAoR, current techniques for searching for two KAoR axis points (e.g., one medial, one lateral) are performed at a high level, where relative motion for all points in the search space is calculated and then the minimum is found. Rather than resolving the points using an orthogonal coordinate system as in the first embodiment (e.g., see FIGS. 3-5), a second embodiment of the present invention utilizes an algorithm that uses a Cartesian coordinate system so that spacing between all points in the search space is uniform. With the orthogonal coordinate system of the first embodiment, spacing between the points increased the further the points got from the origin. That is, the orthogonal coordinate method for defining points in the KAoR search space results in points fanned out radially from the center of the contact pad 42/420. The algorithm in the second embodiment changes how the search space is populated (e.g., to Cartesian coordinates, with an equally spaced grid around the contact pad 42/420). Thus, the change in the algorithm from the first embodiment to the algorithm of the second embodiment comprises of a change from a radial search space to a grid search space for the KAoR. By using a Cartesian coordinate system, the search space is a uniform grid with each point spaced (e.g., 1 mm) apart in the x and y directions of the plane. Thus, the algorithm of the second embodiment modifies how search space points are populated as compared to that in the first embodiment, but the subsequent calculations generally remain the same as in the first embodiment.

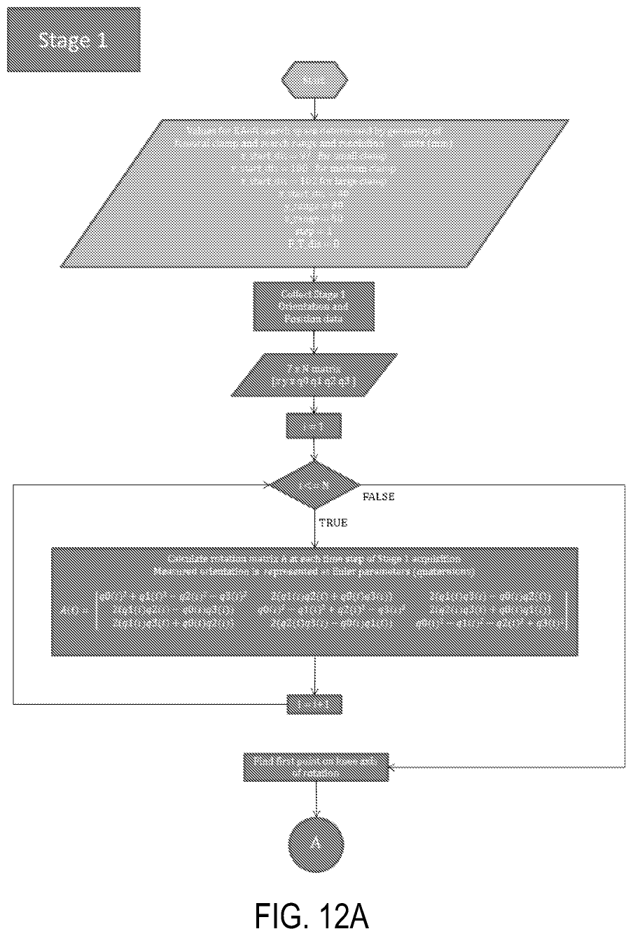

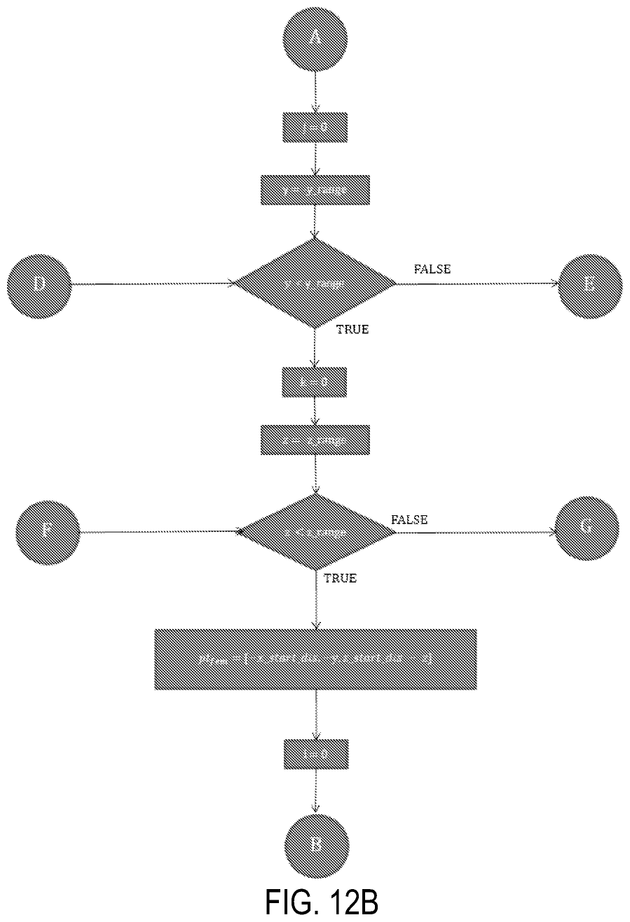

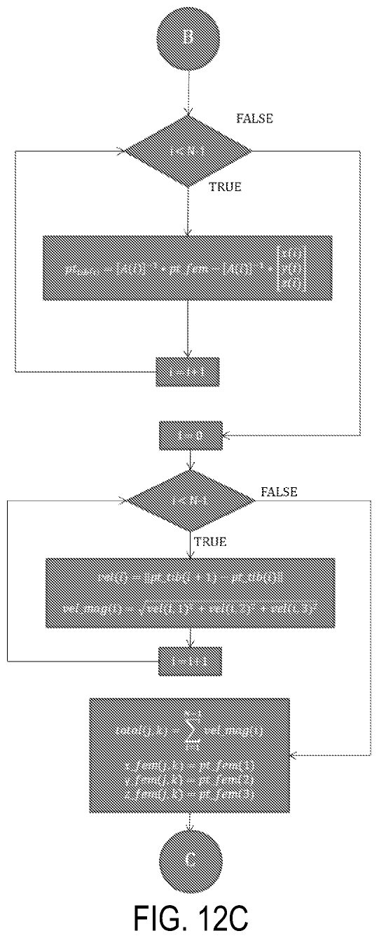

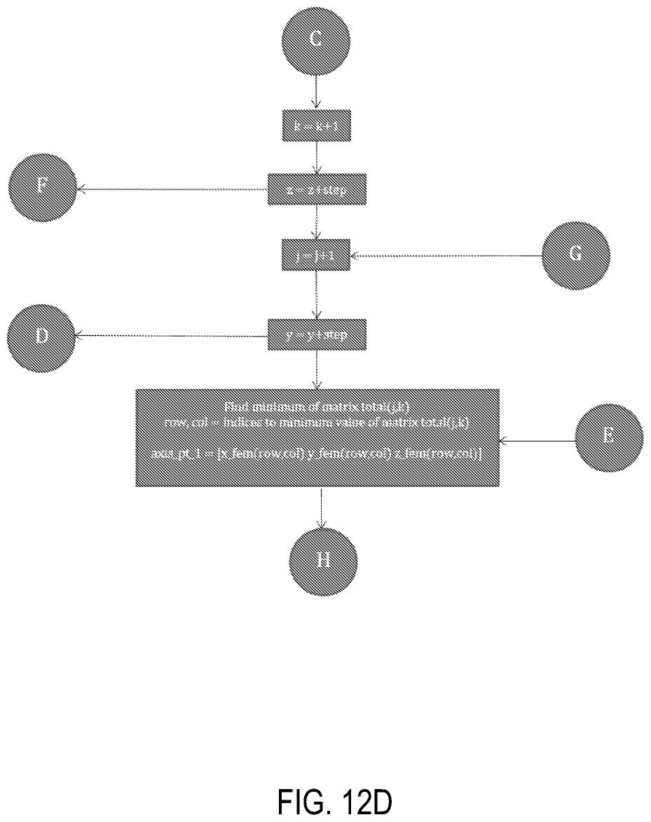

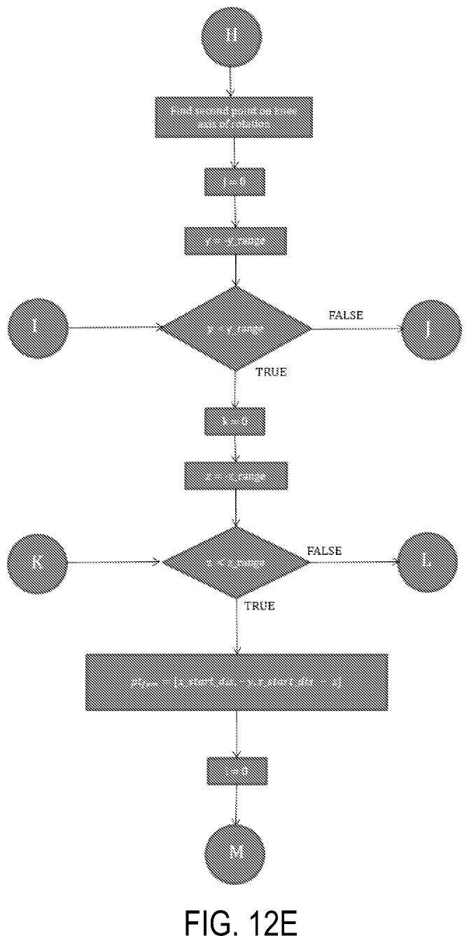

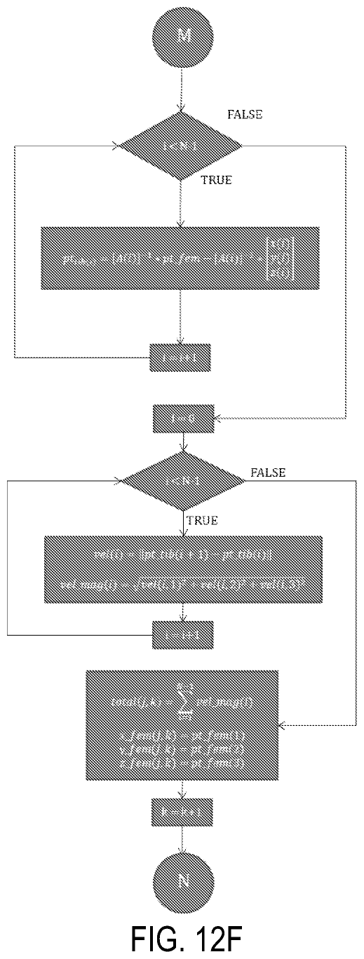

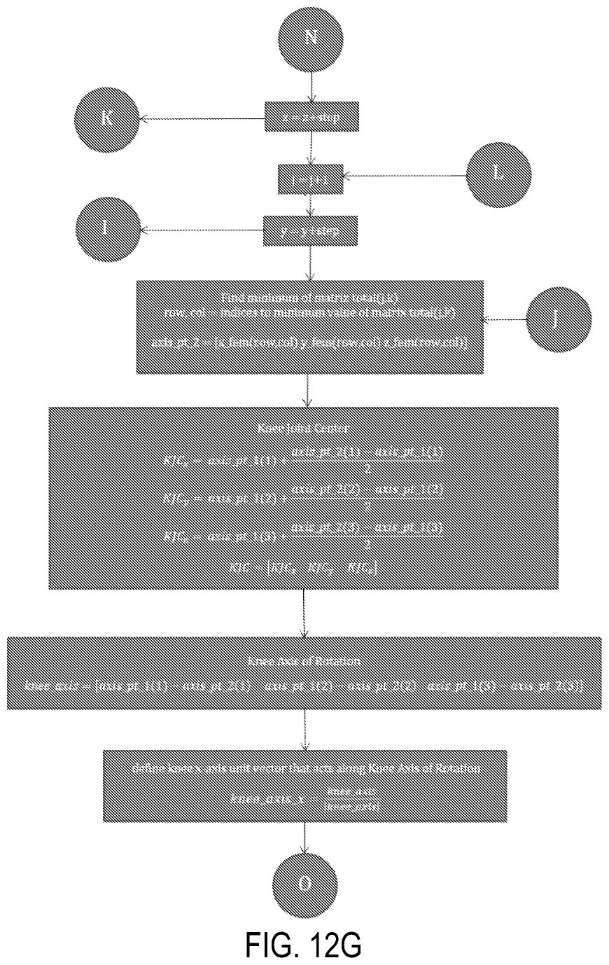

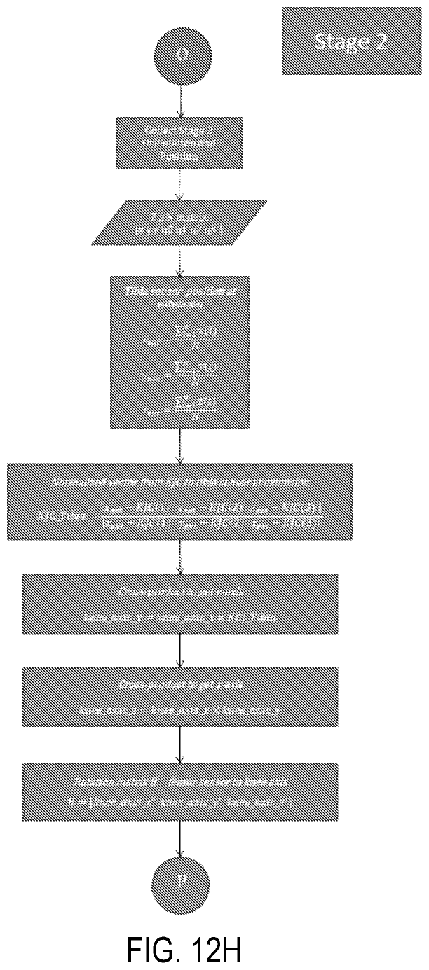

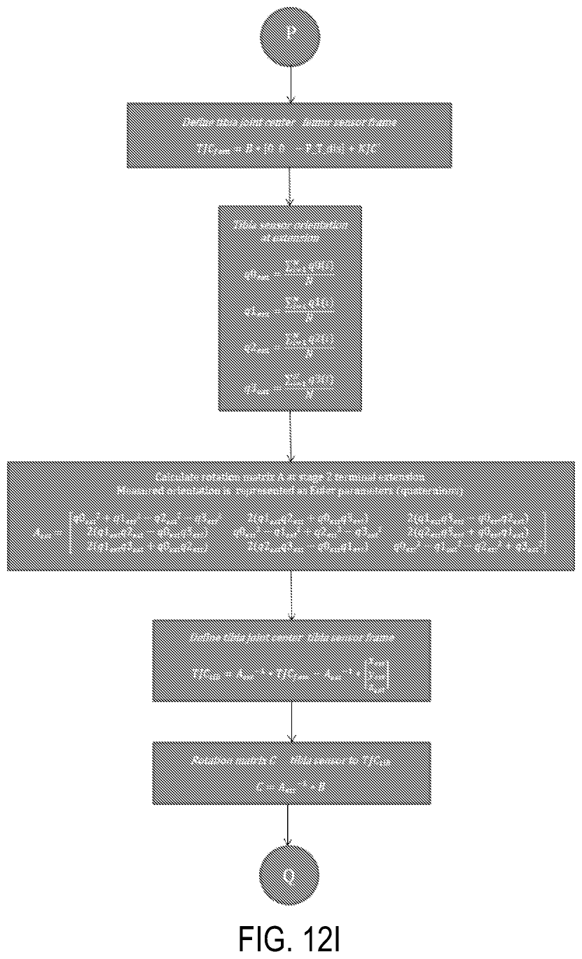

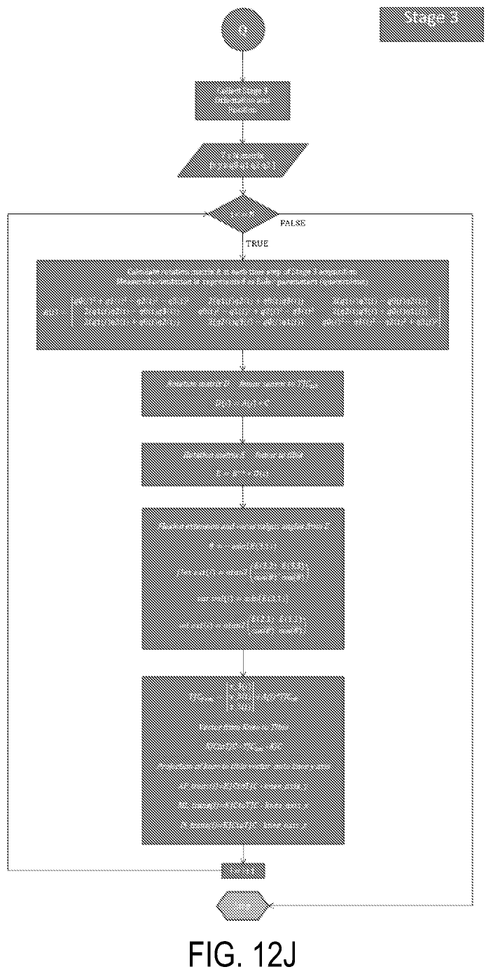

FIGS. 12A-J illustrate a flow chart of the algorithm used in the second embodiment which uses Cartesian coordinates to identify points in the search space (different from the orthogonal coordinate approach in the first embodiment). The algorithm is categorized by three stages (Stage 1, Stage 2 and Stage 3), where FIGS. 12A-12G illustrate Stage 1, FIGS. 12H and 12I illustrate Stage 2, and FIG. 12J illustrates Stage 3. This algorithm may be executed by a computer system (not shown) such as that associated with processor 26 as shown in FIG. 1 that is capable of processing the data acquired by the sensors.

Although the algorithm of the second embodiment is described in connection with the knee arthrometer 200, the knee arthrometer 20 described above can likewise be used in conjunction with the algorithm of the second embodiment. Additionally, the algorithm of the second embodiment can be used for measuring knee laxity as described above in the first embodiment, as well as for measuring dynamic functional tests (e.g., step-up/squat as shown in FIGS. 10 and 11 as well as other functional movements as described above). While the Cartesian-based algorithm of the second embodiment is generally preferable to the orthogonal-based algorithm of the first embodiment, the orthogonal-based algorithm of the first embodiment can be used for dynamic functional tests (e.g., step-up/squat as shown in FIGS. 10 and 11) in addition to knee laxity application as described above.

In Stage 1 of the algorithm of the second embodiment, the determination of the values for the KAoR search space are determined as described above. The rotation matrix “A” as described above is calculated, and points of knee axis rotation are found (see FIGS. 12A and 12E). The ultimate output from Stage 1 is a knee x-axis unit vector that acts along the KAoR (see output “0” in FIG. 12G).

In Stage 2, the rotation matrix “B” as described above and the rotation matrix “A” (at stage 2 terminal extension) are calculated. The ultimate output from Stage 2 is the solution of the rotation matrix “C” as described above.

In Stage 3, the rotation matrix “A” (at each time step of Stage 3 acquisition) is calculated, and the rotation matrix “D” and the rotation matrix “E” as described above are solved. From Stage 3, a vector from knee to tibia is calculated, and a projection of the knee to tibia vector onto a knee axis (x-, y- and/or z-axes) is performed.

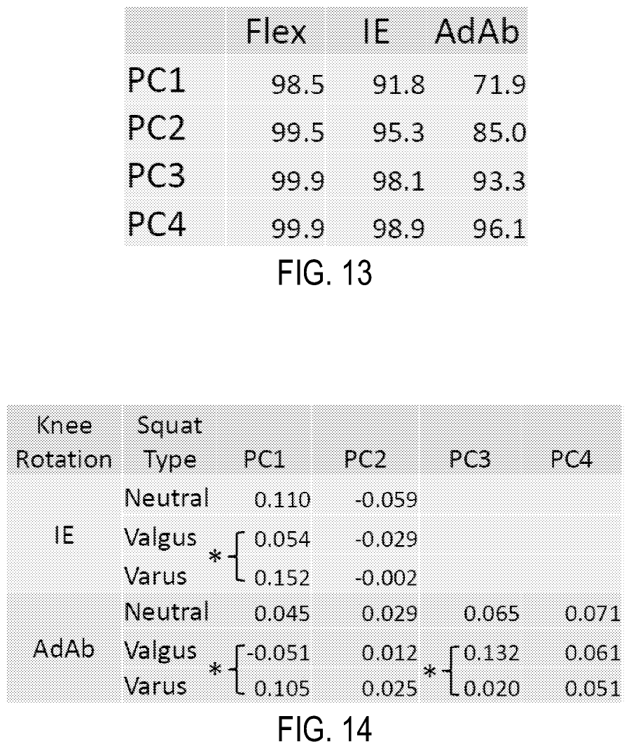

FIGS. 13-18 illustrate experimental results collected from testing using the knee arthrometer 200 and the algorithm of the second embodiment.

As described above, the KAoR is the axis where the tibia rotates around the femur during motion. The KAoR anatomically lies close to an axis in the femur that passes through the medial and lateral epicondyles. Tests conducted with test subjects using knee arthrometer 200 were conducted as follows. The femoral frame 220 was placed on a leg (e.g., 280) of a test subject such that the five (rubber tipped) posts (e.g., 221) were placed around each of the protruding medial and lateral epicondyles of the subject to create a circle target area. The center of the circle target area serves as a starting point for the KAoR search space (see, e.g., Stage 1 in FIGS. 12A-12G). Each point in the search space was defined in femur coordinates and tibia coordinates. Points were tracked during the flexion-extension motion of Stage 1. If the point was found to lie on the KAoR, then relative movement between the femur point and tibia point was minimized.

In testing (e.g., during Stage 3, after defining anatomical coordinate systems in Stages 2 and 3), test subjects wearing the knee arthrometer 200 performed knee movement tasks that included (i) a step-up onto a (e.g., 20 cm) box such as the box 1000 in FIG. 10, and (ii) dual limb squats (DLS) (see, e.g., FIGS. 11A-C) performed in neutral alignment, with knees brought together (Valgus), and with knees outward (Varus). Knee (e.g., right knee) kinematics were measured using electromagnetic position and orientation sensors (e.g., 380, 500) connected to the bones via the femoral frame 220 secured to the distal femur and the tibial frame 240 secured to the anterior crest of the tibia just below the tibial tuberosity.

The algorithm of the second embodiment defines the KAoR based on (e.g., knee flexion-extension) cycles captured over a 6 second period (e.g., in Stage 1). Sensor measurements from a weight bearing anatomical position were used to define any remaining orthogonal knee axes (e.g., during Stage 2). Standing in anatomical position is essentially putting the leg in terminal extension. There are two ways of acquiring the above-described y-axis, depending on whether subject is sitting on a plinth for laxity or standing for dynamic functional tests (e.g., squatting/step-up).

Stage 1 and Stage 2 are basically the same for laxity and dynamic functional measurements, except as follows. For laxity measurements, the test subject typically sits, whereas in the dynamic functional measurements the test subject typically stands. Stages 1 and 2 are used to determine the anatomical coordinate system based on knee flexion/extension, for example. Stage 3 measures motion of the tibia bone relative to the femur bone using the coordinates defined in Stages 1 and 2. For the laxity test, Stage 3 measures passive motion while a therapist manipulates the limb of the test subject (e.g., in the form of a Lachman's test or similar procedures or acquiring the passive envelope of motion for the knee). For the dynamic functional test, tibiofemoral motion is measured during functional tasks as described above (e.g., squat, etc.).

As an example of how the above-described arthrometer device can be used to measure tibiofemoral motion during functional movements, test subjects performed 3 cycles of each task. The entire data collection, including determination of the KAoR, was then repeated resulting in a total of 6 cycles for each task. Total test time for each test subject was less than 15 minutes. Step-up and squat cycles as described above were extracted from the motion data and time normalized. Ensemble averages of 3D knee motion for each subject provided input to a principal component analysis of the three squat tasks. The results from the test subjects (e.g., measured knee rotations during the step-up task) were within one standard deviation of knee rotations measured on patients using known dual-fluoroscopy techniques. The algorithm shown in FIG. 12 may be executed on a computer system such as the system including processor 26 as shown in FIG. 1, or a separate system (e.g., cloud-based).

FIG. 13 illustrates percent variance as explained by principal components (PC) for each of the 3 knee rotations (i) flexion-extension (“Flex”), ii) internal-external (“IE”), and iii) adduction-abduction (“AdAb”). The PCs provide a method to analyze joint angle waveforms and/or a pathway to convert measured tibiofemoral motion into clinically meaningful diagnostics/information by extracting certain signatures in the motion data. For example, for squatting, the first 2 PCs explained 95.2% of the variance for internal-external (IE) rotation. The first 4 PCs explained 96.1% of the variance for adduction-abduction (Ad-Ab).

FIG. 14 illustrates mean PC scores for Neutral, Valgus and Varus squatting (see FIGS. 11A-C). The means of the IE PC1 score between Valgus and Varus squatting were statistically different (P<0.001) as well as Ad-Ab PC1 (P<0.001) and PC3 (P=0.001) scores. Knees were more externally rotated and abducted (tibia relative to femur) during Valgus squatting (see FIGS. 11A-C), whereas knees were more internally rotated and adducted during Varus squatting. From the tests, female knees were found to be more internally rotated and abducted during all squatting tasks, but the differences in PC scores were not statistically significant. In FIG. 14, Valgus/Varus PC scores that were statistically different are indicated with an asterisk.

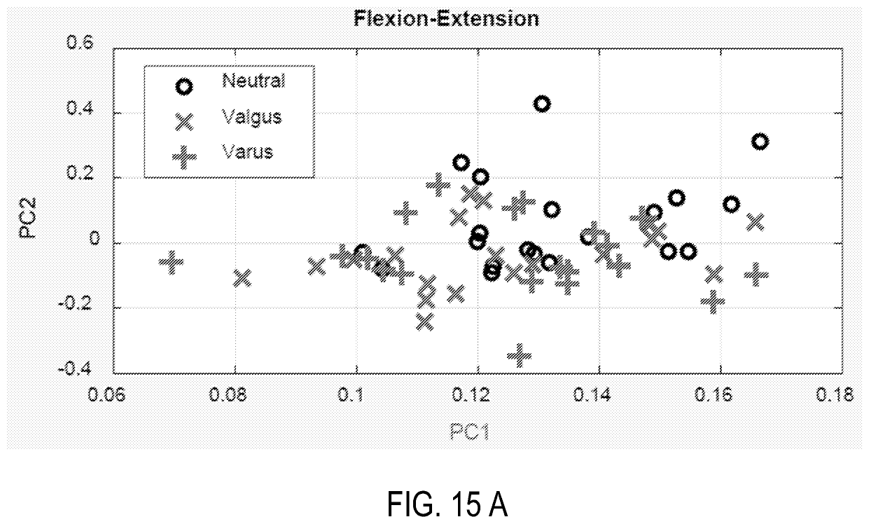

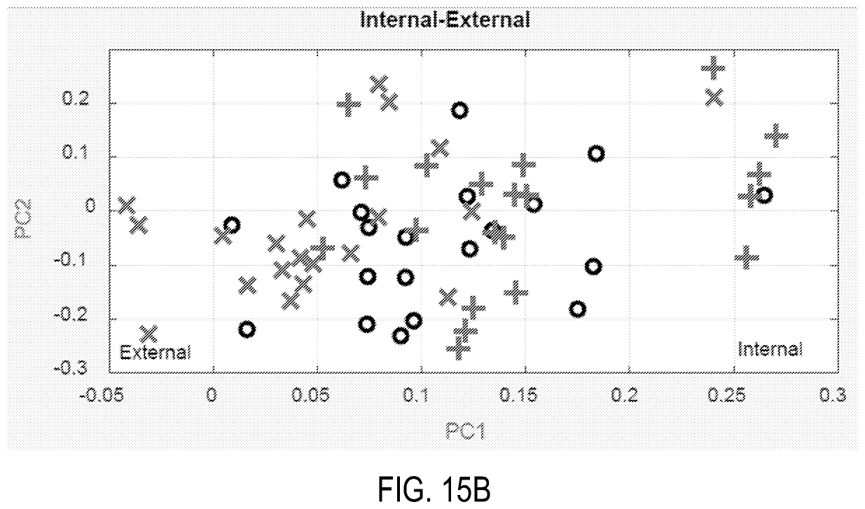

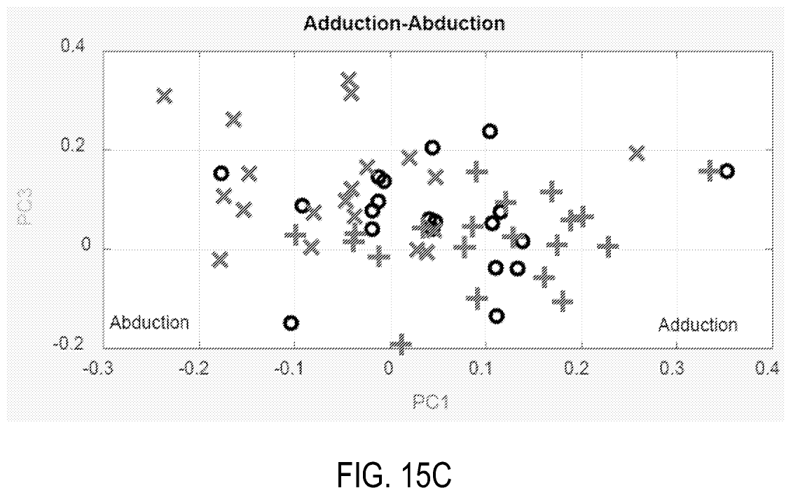

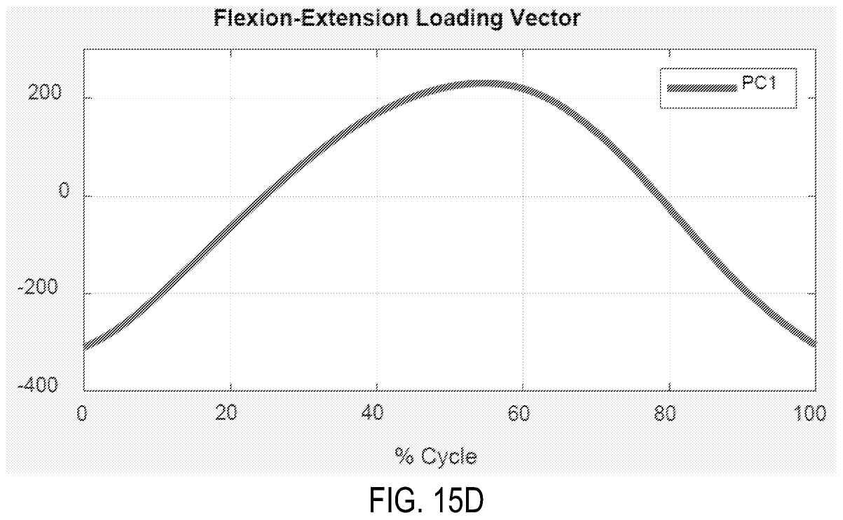

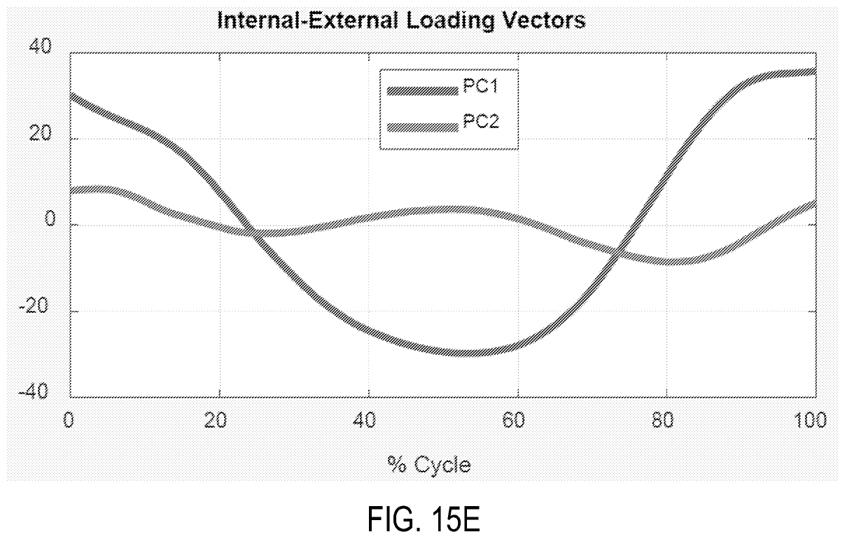

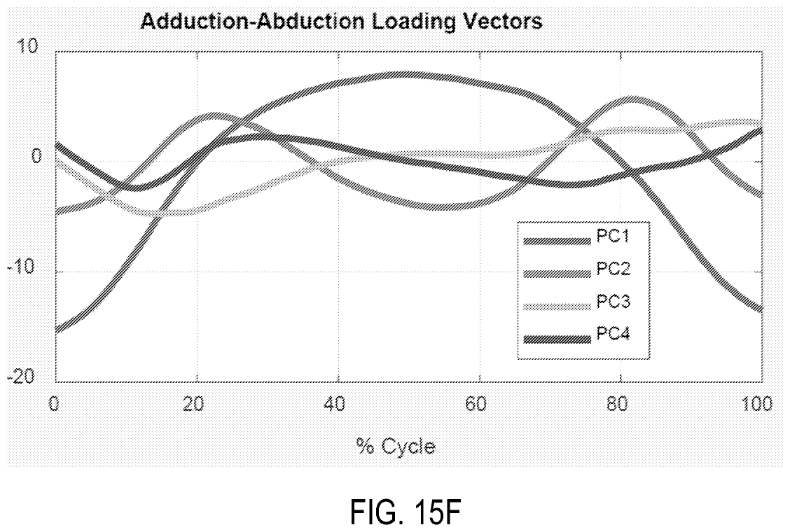

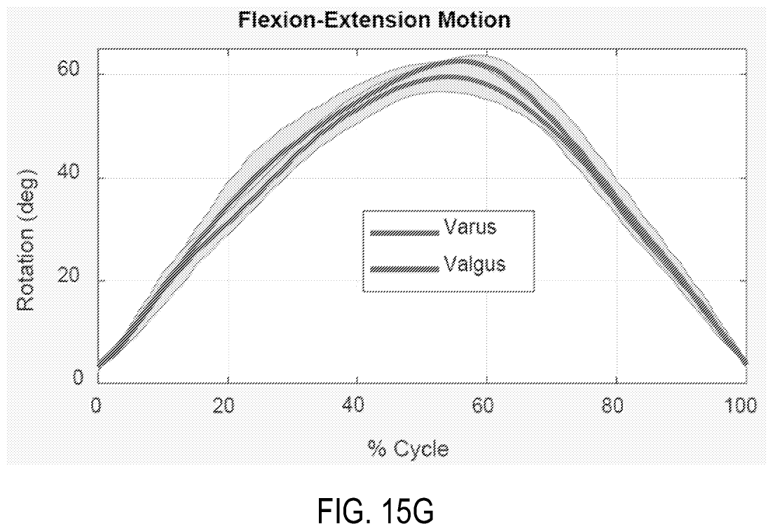

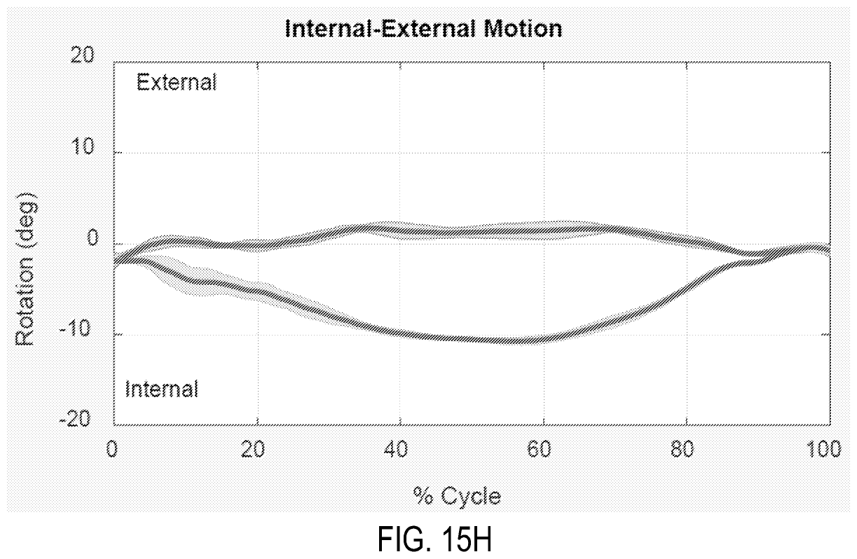

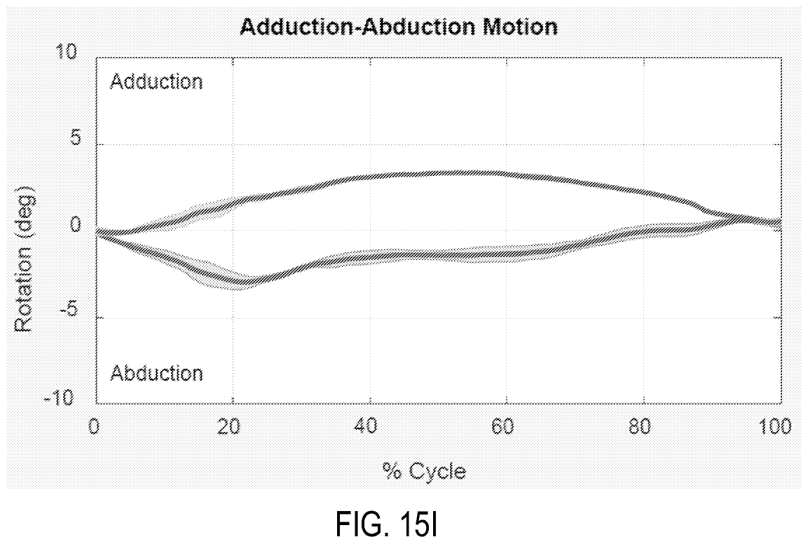

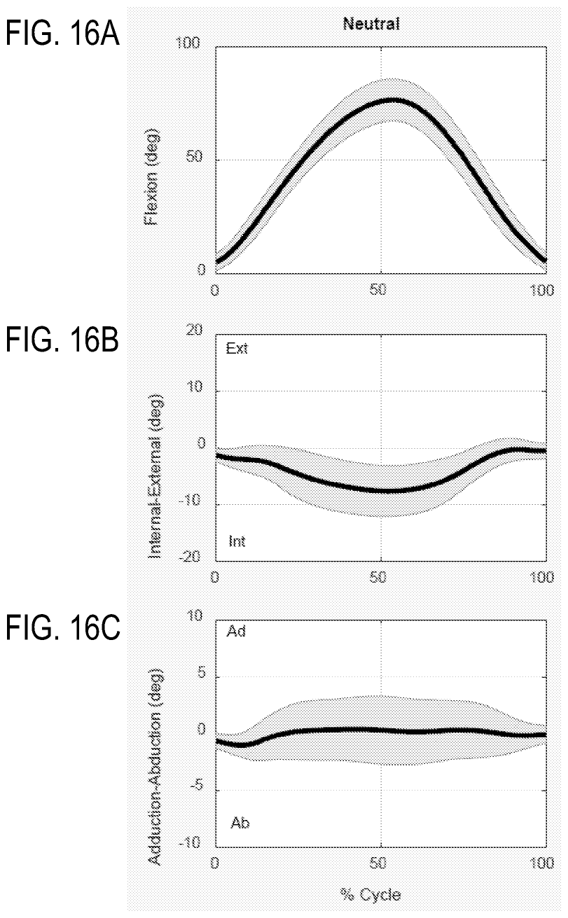

FIGS. 15A-15I, 16A-C, 17A-C, and 18A-C illustrate results from testing of various knee movements of test subjects, such as where knees were more externally rotated and abducted (tibia relative to femur) during Valgus squatting and where knees were more internally rotated and adducted during Varus squatting.

FIG. 15A illustrates Flex PC1 scores versus Flex PC2 scores for the three squatting tasks for neutral, Valgus and Varus knee positions. FIG. 15B illustrates IE PC1 scores versus IE PC2 scores for the three squatting tasks (using the same legend markers shown in FIG. 15A). Positive IE PC1 scores corresponded to tibias that rotated internally during the squat cycle. FIG. 15C illustrates Ad-Ab PC1 scores versus PC3 scores (using the same legend markers shown in FIG. 15A). Positive Ad-Ab PC1 scores correspond to adduction. For example, high values for PC3 adduction/abduction may indicate knee impairment. FIG. 15D illustrates PC loading vectors for Flex waveforms. FIG. 15E illustrates IE waveforms for PC1 and PC2. FIG. 15F illustrates Ad-Ab waveforms during the squatting tasks for PC1-PC4. Shown are the PCs that explain 95% of the variance for each knee rotation. FIGS. 15G, 15H and 15I show tibiofemoral rotations (flexion (“Flex”), internal-external (“IE”) and adduction-abduction (“Ad-Ab”)) over a Valgus squat cycle and Varus squat cycle for one test subject. The test subject performed each squat type 6 times and shown are the average and one standard deviation. FIG. 15G illustrates ensemble averages±one standard deviation of measured rotations for one subject for Flex motion. FIG. 15H illustrates IE motion averages, and FIG. 15I illustrates Ad-Ab rotation during Valgus and Varus squatting. FIGS. 15H and 15I use the same legend markers as FIG. 15G.

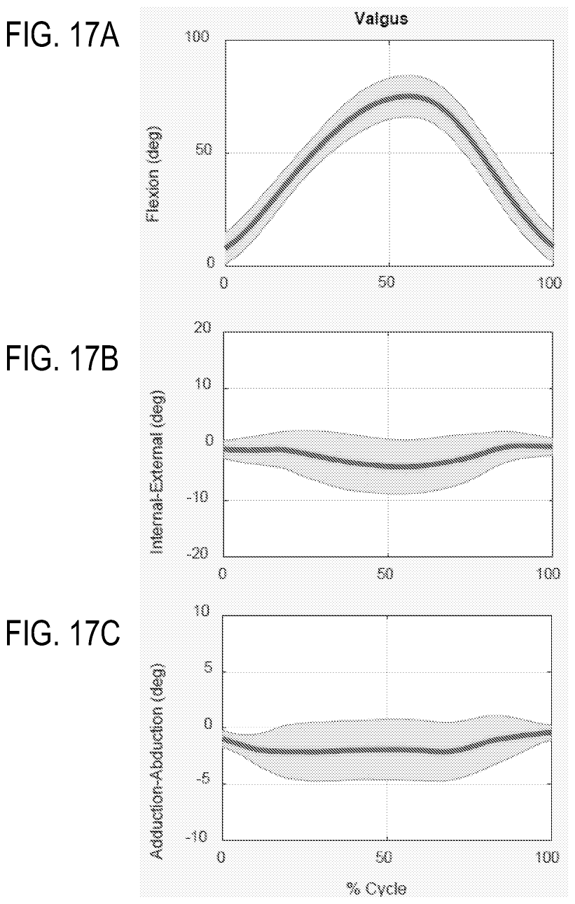

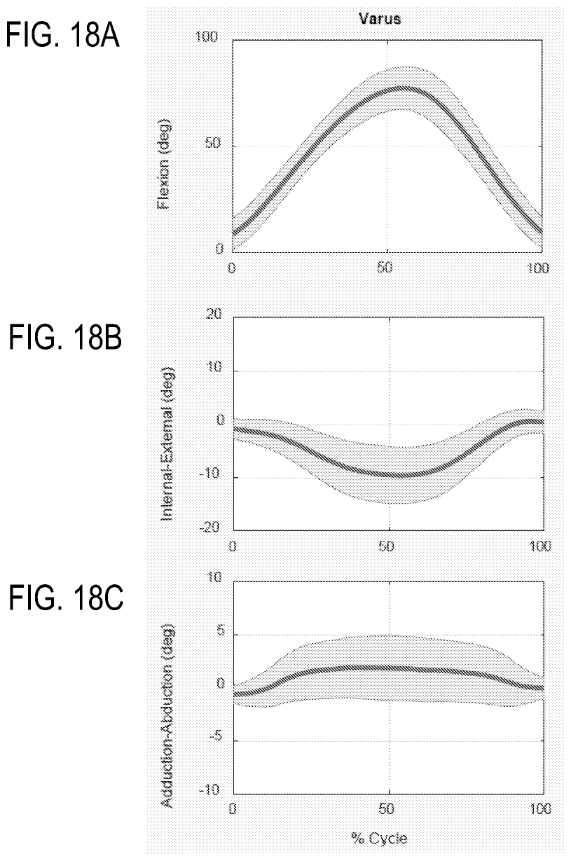

FIGS. 16-18 illustrate knee rotations for the three squatting tasks (neutral, Valgus and Varus, described above), where the mean for 20 test subjects is shown (+/− one standard deviation). FIGS. 16A-C illustrate results from the neutral squatting task described above, FIGS. 17A-C illustrate results from the Valgus squatting task described above, and FIGS. 18A-C illustrate results from the Varus squatting task described above.

In general, IE motion during squatting was dominated by PC1 which peaks at approximately 50% of the squat cycle. IE PC1 scores are greatest for Varus squatting, indicating this task has the largest internal tibia rotation. IE PC1 scores were smallest for Valgus squatting, with some subjects experiencing external rotation (see FIGS. 11A-11C). Ad-Ab PC1 and PC3 scores were significantly different between Valgus and Varus squatting. Ad-Ab PC3 has an abduction peak at ˜15% of the squat cycle that is more pronounced for Valgus squatting. Dynamic leg Valgus during squatting was associated with a reduction in internal rotation with a few subjects exhibiting external rotation. As described above, females were found to be more internally rotated and abducted during squatting activities when compared to males.

From the results, it has been shown that the knee arthrometer 200 and the algorithm of the second embodiment can efficiently and accurately measure tibiofemoral bone motion in a non-invasive, cost effective manner. Differences in knee kinematics were able to be determined in real-time using the knee arthrometer 200 and the algorithm of the second embodiment, representing an improvement over conventional techniques.

In view of the foregoing, it will be seen that the several advantages of the disclosure are achieved and attained. For example, and without limitation, the knee arthrometer 20/200 provides for objective tests of knee laxity and other knee movements described above, as opposed to the current subjective tests for determining knee laxity. The knee arthrometer 20/200 can be used to assess injury to the knee such as tearing of the anterior cruciate ligament. The knee arthrometer 20/200 can replace the Lachman test and/or anterior drawer test with an objective measure. The knee arthrometer 20/200 provides measurement through dynamic 6 degrees-of-freedom motion which is superior to current testing machines which measure only static single degree-of-freedom displacement. These current machines are inaccurate and are not able to quantify dynamic rotational laxity and/or tibiofemoral motion as the knee arthrometer 20/200 is capable of quantifying. The operation of the knee arthrometer 20/200 also is less dependent on sensor placement relative to other existing machines. This provides for increased measurement repeatability (e.g., when measuring knee laxity throughout a treatment regimen).

Other aspects of the invention provide for using the device and techniques described herein for evaluations relating to issues such as (i) muscle spasticity and/or (ii) other impairments such as those associated with (e.g., treatment of) various other disorders (e.g., such as evaluating the impact of ankle foot orthotics on test subjects, where ankle foot orthotics are prescribed to individuals suffering from issues such as foot drop as a result of disorders such as cerebral palsy). For example, muscle spasticity affects millions of people worldwide, but it is very difficult for health care professionals (e.g., physical therapist) to manually assess the severity of a patient's muscle spasticity with accuracy, making it difficult to determine the appropriate treatment. To combat these difficulties, an (e.g., automated) method using (e.g., two) sensors as described herein can be utilized for measurement of a spastic joint angle, thereby making diagnosis and treatment of muscle spasticity easier and more accurate. The Tardieu Scale and Modified Tardieu Scale are clinical measures of muscle spasticity for use with patients with conditions such as neurological conditions, where spasticity is quantified by assessing the muscle's response to stretch applied at given velocities. The quality of the muscle reaction is often velocity dependent. The muscle reaction at specified velocities and the angle at which the muscle reaction occurs are incorporated into the measurement of spasticity using the Modified Tardieu Scale. For example, a conventional assessment using the Modified Tardieu Scale involves manipulating the joint rapidly until the therapist feels a sudden resistance, known as the R1 catch. The physical therapist then eases the joint past its “catching point” slowly. Oftentimes, the physical therapist is trying to determine the joint angle at which the muscle spasticity occurs, but since this joint angle is velocity dependent, it can vary depending on how fast the therapist manipulates the foot. The joint angle can be found by estimating the approximate configuration at which the spasticity occurred and using a goniometer to measure the joint angle at that configuration. However, this method is not very accurate in determining the joint angle, a value that physical therapists use to select and evaluate muscle spasticity treatments. This method is also not able to measure the spastic velocity, a value that potentially could make the assessment more consistent. Accordingly, improvements are needed to improve muscle spasticity assessment.

The device and techniques described herein can realize improvements in muscle spasticity assessment. For example, sensors (e.g., 38, 50) such as those described herein can be used to assess muscle spasticity by conducting a Modified Tardieu Assessment. The algorithms described above in FIGS. 3-5 and 12 can be adapted as necessary in conjunction with the muscle spasticity assessment. Generally speaking, one sensor is securely attached to the frontal, proximal tibia using a securing mechanism (e.g., 46) such as those described herein. Another sensor is securely attached to the dorsal calcaneus using a securing mechanism. Before being attached to the calcaneus, this sensor is used to locate both malleoli. The position of both malleoli is recorded by briefly placing the sensor on each malleoli and collecting data. Establishing the location of the malleoli allows for the flexion/extension axis of the talocrural joint to be established (e.g., for muscle spasticity assessments about the ankle, the anatomical ankle flexion axis can be determined by physically probing the medial and lateral malleoli of the ankle).

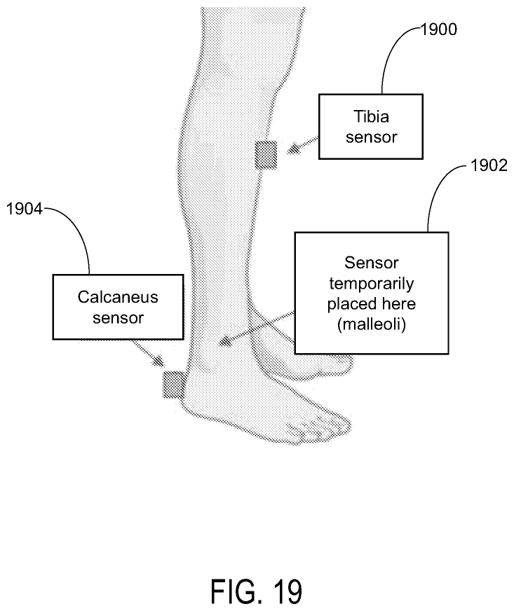

FIG. 19 shows such sensor positioning and placement, wherein the tibia sensor is positioned at location 1900 (e.g., at the tibia), and the other sensor is initially positioned at the malleoli at location 1902 before finally being secured at the calcaneus at location 1904 (although only one location 1902 for the malleoli is shown, the location 1902 includes both malleoli). The other sensor may also be referred to as an auxiliary/third sensor. More specifically, the spasticity assessment involves (i) securely attaching the tibia (aka global) sensor, (ii) placing the calcaneus (aka local) sensor on the lateral malleoli, holding for 10 seconds and collecting data, (iii) placing the calcaneus sensor on the medial malleoli, holding for 10 seconds and collecting data, (iv) securely attaching the calcaneus sensor, (v) placing the subject's foot in a neutral position and collecting data for 10 seconds; (vi) and performing a Modified Tardieu Assessment while collecting data. From the assessment, four text files are generated and are later inputted into code to be analyzed (discussed below). Of note, the location of the malleoli is necessary to establish the ankle joint axis of flexion/extension, and it is necessary that the sensors are placed at the tibia and calcaneus to get the relative motion during the Modified Tardieu Assessment. Although not shown, the knee arthrometer described above may be used with the embodiment shown in FIG. 19. For example, the femoral frame 22/220 (with or without its own sensor) may be used in the embodiment shown in FIG. 19 (see FIGS. 2, 6, 7 and 9, for example). The tibial frame 24/240 and/or other securing mechanisms (e.g., such as 430, 46/460, etc.) may be used in the embodiment shown in FIG. 19 to provide for securing of the sensors, such as at positions 1900, 1902 and 1904 (see FIGS. 2 and 6-9, for example). For example, the tibial frame 24/240 may be used to secure a (tibial) sensor at position 1900, and a securing mechanism such as a strap (e.g., 430, 46/460) may be used to (temporarily) secure a sensor(s) at positions 1902 and 1904. However, a more rigid securing mechanism than a flexible/expandable/elastic strap may be used to secure a sensor at position 1904. Such rigid securing mechanism may include a frame similar to frames 22/24/220/240, but designed for attachment at the calcaneus. The sensors may be described as first, second, third, etc. sensors. For example, the femur sensor may be a first sensor, the tibia sensor may be a second sensor, and the sensor used for the malleoli/calcaneus may be a third sensor. Also, the leg may be referred to as comprising the femur and tibia, as well as comprising the femur, tibia, ankle (e.g., malleoli), and foot (e.g., calcaneus), including portions such as the calf and hamstring.

For comparison, in connection with the above-described knee embodiments/measurements, for the knee motion (e.g., flexion extension to determine flexion axis (KAoR)), accurate determination of KAoR is desired for repeatable and anatomically accurate measurement of knee arthrokinematics with respect to plane motion (e.g., particularly with respect to frontal and transverse plane motion). While the KAoR roughly aligns with the epicondyles, these are difficult to accurately palpate. However, for the ankle, malleoli are easy to palpate and the flexion axis determined with probing the malleoli is sufficient.

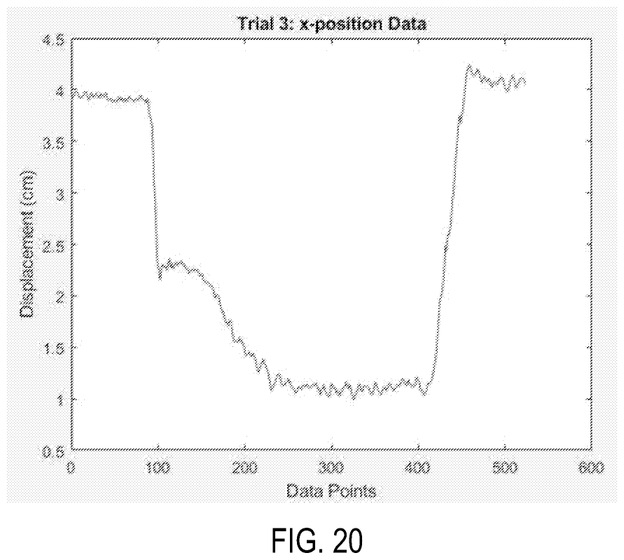

FIG. 20 is a graph that plots the amount of data points on the x-axis and displacement (in cm) on the y-axis. As shown in FIG. 20, there is a telltale “signature” in the position and joint angle data at the moment of spasticity (e.g., the R1 catch). That is, FIG. 20 demonstrates how evident the R1 catch is in the collected (sensor) data. The initial sudden drop is when the rapid force was applied to the sole—where that linear line suddenly ends, is where the R1 catch occurs. The slope that follows is when the therapist applies slower pressure to ease past that “catch.” The presence of this signature allows the code to easily and reliably calculate the spastic joint angle and velocity.

The above-described algorithms (e.g., see FIGS. 3-5 and 12) can, for example, be modified as necessary for such spasticity parameters and implemented using certain software such as MatLab and the like. The code inputted into the software may comprise four inputs: a lateral malleoli text file; a medial malleoli text file; a neutral position text file, and a Modified Tardieu Assessment text file. The inputted text files may have three position columns and four orientation columns. The outputs may comprise two outputs: joint angle and joint velocity. These outputs give the physical therapist the units of the values as well as explain the connection between the joint angle sign and anatomical position. For example, the joint angle may be provided in units of degrees while the velocity may be provided in units of deg/s, and the positive joint angle may be dorsiflexion while the negative joint angle may be plantar flexion. Execution of the code may comprise various stages, such as Stage 1, Stage 2 and Stage 3 (see, e.g., FIGS. 3-5 and 12 and the related algorithm discussion). For example, with respect to the ankle, malleoli points could be incorporated into Stage 1, such points being gathered at center of the malleoli to determine an axis of rotation. Parameters relating to the foot being in a neutral position could be incorporated in Stage 2, for example. And Stage 3 could generally be the same as described above, but with the addition of a velocity calculation for flexion as well as the algorithm for detecting the “catch” signature. For example, in Stage 1, the code reads in the data from the two malleoli text files, and then finds, for example, a talocrural joint center, a plantar/dorsiflexion axis, and a unit vector. In Stage 2, the code reads in the data from the neutral position text file, and creates, for example, a rotation matrix that gives the relative angles of the calcaneus with respect to the tibia. Three vectors are then defined in the ankle location to create a (e.g., calcaneus) coordinate system, for example. Such calcaneus coordinate system is coincident to the tibia coordinate system when the foot is in the neutral position, and thus the relative rotation to the neutral position can be determined while the assessment is being performed. Two rotation matrices are then created and are assumed to behave as rigid bodies, for example. The relative motion of these two rigid bodies can be calculated after the Modified Tardieu Assessment is performed. In Stage 3, the code reads in the data from the Modified Tardieu Assessment text file. Using the quaternions, another rotation matrix is created, similar to the first rotation matrix in Stage 2, for example. Using this rotation matrix, the relative rotation of the calcaneus to the tibia can be calculated. Then, using this rotation, the angle about the ankle joint axis can be calculated. This gives the joint angle for every data point during the assessment. The velocity for every data point during the assessment is then calculated using the joint angle data and the sampling rate of the sensors, for example. The joint angle and velocity are then plotted on the same graph and scaled appropriately.

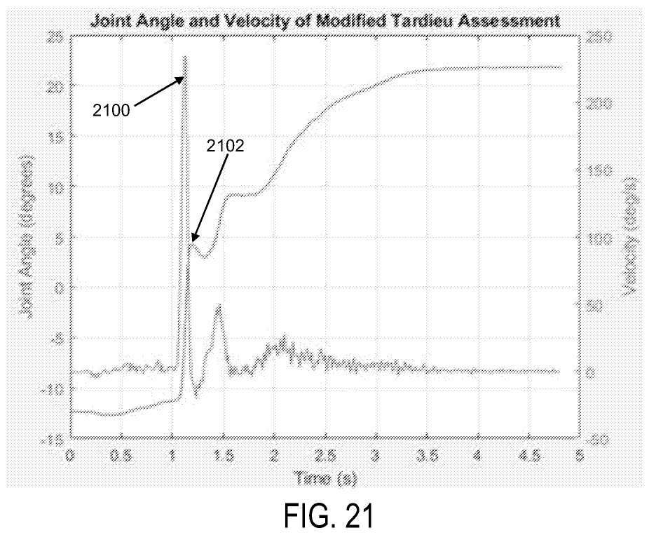

FIG. 21 depicts a graph that is a representative plot reflecting an assessment conducted using the above-described techniques, with the x-axis comprising time (s), the left side y-axis comprising joint angle (degrees), and the right side y-axis comprising velocity (deg/s). The peak 2100 demonstrates the spastic velocity and the peak 2102 demonstrates the spastic joint angle. FIG. 21 thus represents the peak velocity and spastic joint angle as calculated and outputted. In FIG. 21, the velocity line (e.g., the line comprising 2100) begins steadily at zero before the assessment begins. It then raises rapidly as a force is quickly applied to the foot. The velocity reaches its peak value just before the spastic muscle catch and decelerates rapidly as the muscle resists any more dorsal motion. The second, smaller peak in velocity occurs as the physical therapist eases the foot past the spastic muscle catch. The velocity then returns to zero as the assessment ends. The joint angle line (e.g., the line comprising 2102) begins steadily at approximately −12 degrees. The negative angle indicates that the foot is in a slight plantarflexion position before the assessment begins. As the physical therapist applies a force to the foot, the ankle crosses over from plantarflexion to dorsiflexion and the angle becomes positive. The first peak on the joint angle graph gives the spastic joint angle. The maxima of this peak occurs when the velocity is zero because the foot has resisted any further motion. The second peak occurs as the physical therapist slowly eases the foot past that angle of muscle spasticity. The foot then slowly eases to full dorsiflexion as the assessment concludes.

Experimental results include the following: assessing multiple trials, the average spastic joint angle for a single individual was 4.663 degrees (dorsiflexion). These values are similar to the estimated joint angle as estimated by a physical therapist (e.g., a joint angle of 5 degrees (dorsiflexion) given by a physical therapist that performed the assessment). The average spastic velocity for the test subject was 226.8 degrees per second. There was a clear signature of the spastic joint angle and velocity in each of the trials performed on a single subject. This clear signature allowed for reliable identification of the joint angle and velocity within the code. This method of determining spastic joint angle and velocity was able to reliably produce a joint angle similar to the one estimated by the physical therapist. It was also able to consistently measure a spastic joint velocity. Because of this, the two-sensor method of determining spastic joint angle and velocity represents a replacement option for a conventional Modified Tardieu Assessment. This method can identify the moment of muscle spasticity, output the spastic joint angle and velocity, and produce a graph of these values, and therefore can assess abnormal muscle reaction using electromagnetic sensors such as those described herein. Placement of the sensors via a rigid attachment system is envisioned, as well as adaptation for different spastic muscles other than the gastrocnemius.

Thus, while muscle spasticity affects millions of people worldwide, the device and method disclosed herein can aid in the assessment and treatment of these people. The device and method disclosed herein can measure and output the joint angle and velocity at the moment of muscle spasticity, in a manner consistent with estimated joint angle values given by a physical therapist, thereby representing an improvement over conventional techniques (of note, the Modified Tardieu assessment is often used to evaluate the efficacy of drugs used to suppress spasticity, such as Botox).

In addition to the above-described practical applications, the device and methods disclosed herein can also be implemented for use in evaluating the effect of an ankle foot orthotic (AFO) on tibiofemoral motion during certain tasks, such as the above-described step-up task using a step-up box. Ankle foot orthoses are commonly prescribed to improve gait in populations such as those with cerebral palsy and other like disorders causing certain (e.g., knee) impairments. An ankle foot orthotic is a support intended to control the position and motion of the ankle, compensate for weakness, or correct deformities, and can be used to support weak limbs, or to position a limb with contracted muscles into a more normal position. An ankle foot orthotic can also be used to control impairments such as foot drop, known to be caused by a various neurologic and musculoskeletal disorders. An ankle foot orthotic supports the lower limb in cases of ankle or knee weakness or spasticity. Reduction or elimination of (e.g., talocrural) motion can be obtained as well as a reduction in (e.g., subtalar) motion. Moreover, an ankle foot orthotic can be modified to reduce tone or maintain neutral alignment and can further minimize varus/valgus with the addition of additional features such as straps/tabs (e.g., a dynamic force strap and/or medial/lateral tabs or other securing mechanism consistent with those described herein). The overall goal of an ankle foot orthotic is to stabilize the foot and ankle and provide toe clearance during the swing phase of gait, which helps decrease the risk of catching the toe and/or falling. A typical ankle foot orthotic creates an L-shaped frame around the foot and ankle, extending from just below the knee to the metatarsal heads of the foot.

However, currently there is limited evidence available on the long term effects of ankle foot orthoses prescriptions or the possible effects of limiting foot and ankle motion on the tibiofemoral joint. This is an important consideration as adults with cerebral palsy are more likely to experience issues such as knee pain and osteoarthritis, resulting in earlier than normal loss of function. The device 20/200 and associated techniques as described herein can be used in conjunction with an ankle foot orthotic in order to investigate the effects of an ankle foot orthotic on patients. The above-described algorithms (see FIGS. 3-5 and 12) can be adapted as necessary for use in conjunction with ankle foot orthotic testing.

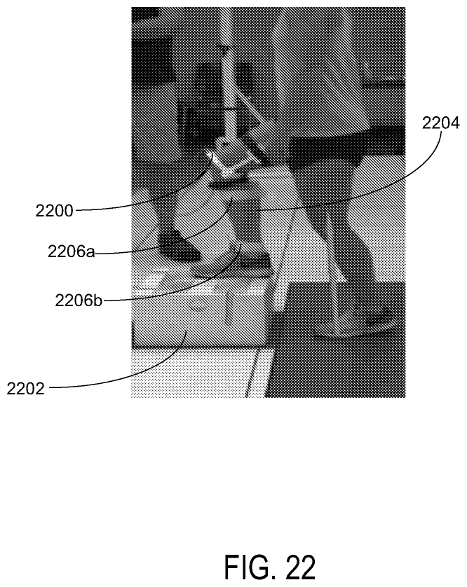

FIG. 22 shows a sample test set-up using a device 2200 (similar to 20/200 as described herein) and a step-up box 2202 similar to that described herein (e.g., 1000 in FIG. 10). In FIG. 22, the test subject is wearing the device 2200 in conjunction with an ankle foot orthotic 2204 (e.g., a solid ankle foot orthotic). This ankle foot orthotic 2204 may be custom fabricated out of molded plastic or other like material (e.g., metal, leather or other composites such as a carbon composite), or may be an off-the-shelf variant, such as an Optek full sole solid ankle foot orthotic, constructed of 3/16″ polypropylene and secured with pre-tibial and ankle fastening straps 2206a and 2206b, similar to the above described securing mechanisms (e.g., 430). The test subject performs step-up maneuvers indicated by the arrows shown in FIG. 22. The testing may comprise three trials of step-up on box 2202, where box 2202 may have a step of a certain height (e.g., an 8″ step), and may comprise the test subject (i) wearing a shoe-only and (ii) wearing a shoe with ankle foot orthotic. Tibiofemoral motion may be collected using electromagnetic motion sensors as those described herein attached to custom made fixtures (consistent with those described herein) secured to the proximal tibia and distal femur. A custom algorithm along the lines described herein (see FIGS. 3-5 and 12) may be used to define the knee axis of rotation based on knee flexion extension cycles performed by the test subject. The outputs of the sensors of device 2200 may include rotations of the tibia relative to the femur in sagittal, frontal, and transverse planes. Ensemble averages of three step-up trials can be calculated, and independent tests can compare the extent of motion and discrete values at 10% increments of the step-up cycle, for example. A Benjamini-Hochberg procedure can be used to control false discovery rate associated with multiple comparisons. In one experiment, participants included n=29 healthy adults (female=19, age=24.4+/−4.5 years, mass=66.8+/−12.8 kg, height=171.8+/−11.1 cm). In general, the participants demonstrated increased abduction and external rotation at the tibiofemoral joint in the shoe with ankle foot orthotic test condition with step-up.

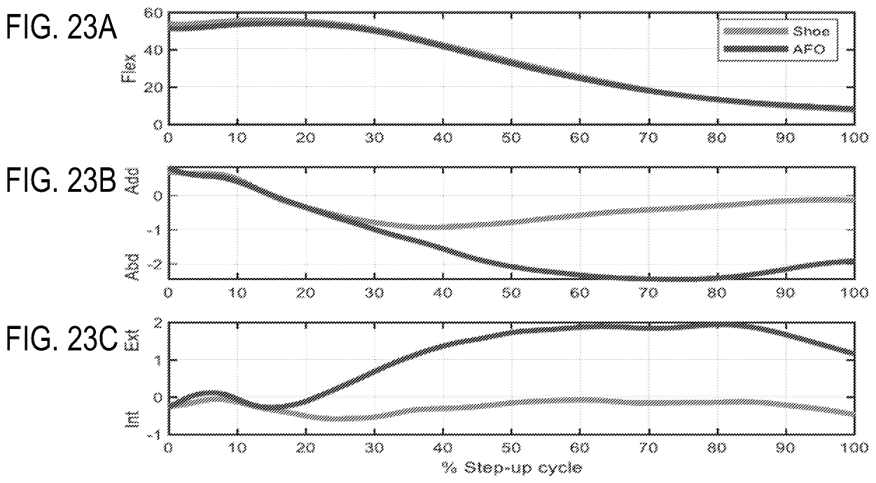

FIGS. 23A-23C show results from the experiment(s) described above in FIG. 22, conducted using the above-described device and methods. FIGS. 23A-23C show mean tibiofemoral motion in sagittal, frontal, and transverse planes in shoe-only and shoe with ankle foot orthotic conditions, where positive values are adduction in the frontal plane and external rotation in the transverse plane. FIGS. 23A, 23B and 23C each have their x-axis representing % Step-up cycle, where FIG. 23A plots flexion on the y-axis, FIG. 23B plots abduction and adduction on the y-axis, and FIG. 23C plots internal and external rotation on the y-axis (e.g., similar to the knee rotations described in connection with FIGS. 13 and 14 above, for example).

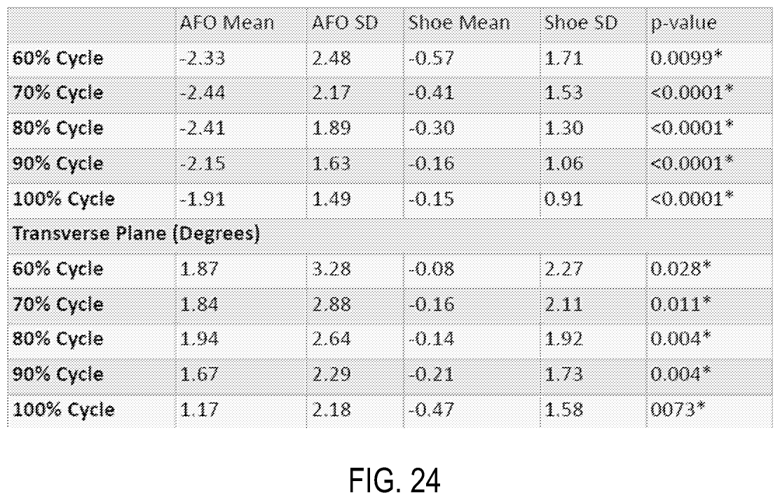

FIG. 24 illustrates data regarding statistically significant discrete ROM data points in the step-up cycle frontal plane (degrees) with respect to various percent cycles for the frontal and transverse planes. FIG. 24 includes mean and standard deviation data for both the shoe-only test and the shoe with ankle foot orthotic test for cycles ranging from 60%, 70%, 80%, 90% and 100%, including p-values for each. Discrete range of motion values from 60 to 100% of the step-up cycle were significantly different in both transverse and frontal planes.

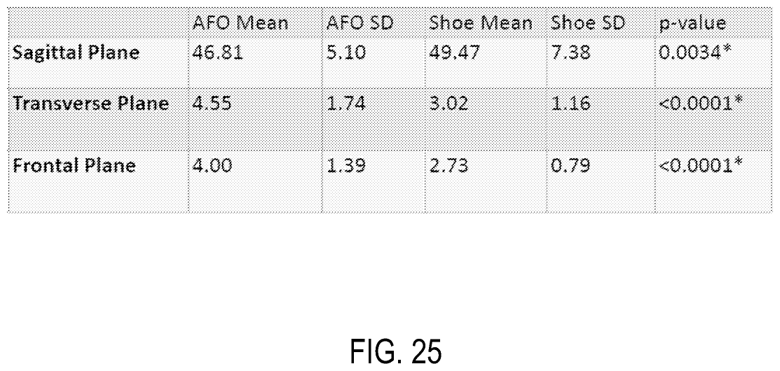

FIG. 25 illustrates data regarding extent of motion (degrees) for the sagittal, transverse and frontal planes, and includes mean and standard deviation data for both the shoe-only test and the shoe with ankle foot orthotic test, as well as p-values for each of the sagittal, transverse and frontal planes. Extent of motion was significantly greater in the shoe with ankle foot orthotic condition in the frontal and transverse planes and less in the sagittal plane.

From the experimental data shown in FIGS. 23-25, the device and methods described herein are able to show that limiting ankle and foot motion with a solid ankle foot orthotic impacts tibiofemoral motion during step-up, and thus the device and methods described herein can be used as a viable tool for evaluating the effect of ankle foot orthotic characteristics on tibiofemoral motion, which is important in evaluating whether ankle foot orthotics may contribute to joint degeneration associated with knee pain and osteoarthritis. In other words, the influence of the AFO on the knee should be a consideration during the fitting of AFOs, and the system and method described herein provide for a means for assessing AFO influence on knee motion. Thus, the device and methods described herein provide for a manner in which to better understand the impact of ankle foot orthotic prescriptions on tibiofemoral motion, and can be used, for example, to optimize ankle foot orthotic prescriptions and wearing schedules of a patient.

In view of the foregoing, it will be seen that the several advantages of the disclosure are achieved and attained, including a portable, inexpensive and quick way to measure, for example, arthrokinematics (e.g., tibiofemoral arthrokinematics), where dynamic joint (e.g., knee) motion can be measured during functional weight bearing tasks. The above-described algorithm(s) allow for quick identification of anatomically-based axes and coordinates. The above-described systems and methods allow for analysis of functional movement during functional tasks including but not limited to stepping tasks and other tasks (e.g., walking), and measurements of joint motion (e.g., including motion at the knee, ankle, and the like). Parameters such as joint angle and velocity at which muscle spasticity occur (e.g., including calf and/or hamstring spasticity) can be measured, for example, at the knee. For example, the ankle can be used to assess calf muscles, while knee flexion/extension can be used to assess hamstring spasticity.

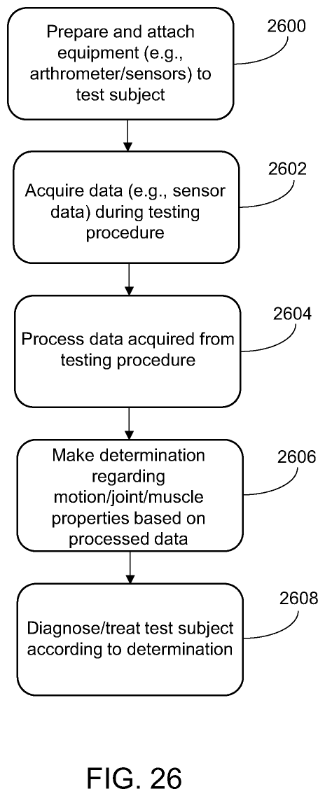

FIG. 26 depicts a sample methodology encompassing the techniques described above in the various embodiments. At step 2600 the equipment (e.g., computer system, software/code, etc.) is prepared and the equipment to be placed on the test subject is attached to the test subject (e.g., the above-discussed frames, straps, etc. of the arthrometer and the sensors are positioned according to the techniques described above). At step 2602 the test subject is positioned for the conducting of a test procedure as described above (such as the various functional tasks and/or other examination techniques conducted by a therapist, such as the therapist moving portions of the leg relative to one another) and the data obtained from the sensors as a result of the movement of the test subject's corresponding body part(s) is acquired. At step 2604 the acquired sensor data from the performed testing procedure is processed. For example, such processing may include processing via the techniques described above in Stage 1, Stage 2 and Stage 3 as well as any other processing necessary for analysis/use of the data consistent with the techniques described herein. At step 2606 a technician/therapist (or software program that is trained/programmed to make a determination based on known markers) may analyze the result(s) generated from the above-noted processing of the data, and can make a determination as to what the data suggests. For example, the processed data may produce a result indicative of a certain physical impairments (e.g., knee impairment) and/or other various properties of the joints/muscles of the overall leg (e.g., knee, ankle, foot, calf, hamstring, etc.) as described above. At step 2608, a technician/therapist (or software program) may establish a diagnosis and/or treatment based on the determination made from the analysis of the processed data.

For all of the above-described embodiments and usages, the code and/or obtained sensor data may be stored in a memory of the above-described computer system, and/or in a remote (e.g., cloud) storage system (e.g., in a dedicated database or other centralized storage mechanism). The raw and/or processed sensor data and/or any related graphical or other representations of the data may be processed by the above-described computer system or the like and output for display on a display device such as a TV, monitor, mobile device (e.g., mobile phone or tablet) and the like such that a technician/practitioner/evaluator/therapist/user can view and/or manipulate the data (e.g., the data may be presented in a visual format for presenting certain aspects of the test results, for example as shown in the applicable above-noted figures). For example, a display monitor may be connected (e.g., wired or wirelessly) to the above-described computer system to provide a visual output on the computer system. The computer system may have an operating system with a graphical user interface capable of being used by a user to (i) input, view, execute and/or manipulate the above-described computer code and/or (ii) process the obtained sensor data and any related graphical representations of such data in the manners described above. The operating system may be capable of running software applications such as those described above (e.g., MatLab and the like) for carrying out the above-described techniques and also any necessary post-processing and/or outputting of the obtained sensor data for viewing, such as for viewing by a therapist that is treating/diagnosing a patient/test subject. Additional software for other code/data manipulations and/or for generating other visuals relating to the data may also be present on the computer system.

In the present disclosure, all or part of the units or devices of any system and/or apparatus, and/or all or part of functional blocks in any block diagrams and flow charts may be executed by one or more electronic circuitries including a semiconductor device, a semiconductor integrated circuit (IC) (e.g., such as a processor), or a large-scale integration (LSI). The LSI or IC may be integrated into one chip and may be constituted through combination of two or more chips. For example, the functional blocks other than a storage element may be integrated into one chip. The integrated circuitry that is called LSI or IC in the present disclosure is also called differently depending on the degree of integrations, and may be called a system LSI, VLSI (very large-scale integration), or ULSI (ultra large-scale integration). For an identical purpose, it is possible to use an FPGA (field programmable gate array) that is programmed after manufacture of the LSI, or a reconfigurable logic device that allows for reconfiguration of connections inside the LSI or setup of circuitry blocks inside the LSI. Furthermore, part or all of the functions or operations of units, devices or parts or all of devices can be executed by software processing (e.g., coding, algorithms, etc.). In this case, the software is recorded in a non-transitory computer-readable recording medium, such as one or more ROMs, RAMs, optical disks, hard disk drives, solid-state memory, servers, cloud storage, and so on and so forth, having stored thereon executable instructions which can be executed to carry out the desired processing functions and/or circuit operations. For example, when the software is executed by a processor, the software causes the processor and/or a peripheral device to execute a specific function within the software. The system/method/device of the present disclosure may include (i) one or more non-transitory computer-readable recording mediums that store the software, (ii) one or more processors (e.g., for executing the software or for providing other functionality), and (iii) a necessary hardware device (e.g., a hardware interface).

The embodiments were chosen and described in order to best explain the principles of the disclosure and its practical application to thereby enable others skilled in the art to best utilize the disclosure in various embodiments and with various modifications as are suited to the particular use contemplated. Aspects of the disclosed embodiments may be mixed to arrive at further embodiments within the scope of the invention.

As various modifications could be made in the constructions and methods herein described and illustrated without departing from the scope of the disclosure, it is intended that all matter contained in the foregoing description or shown in the accompanying drawings shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. A joint arthrometer for measuring arthrokinematics comprising:
a femoral frame attachable to a leg of a test subject about a distal femur of the leg, the distal femur having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion configured to contact the leg about the lateral epicondyle, the second arm portion configured to contact the leg about the medial epicondyle, the connecting portion connecting the first and second arm portions, the femoral motion sensor coupled to the femoral frame and configured to measure motion with six degrees of freedom and output femoral motion data associated with the measured motion; and
a tibial frame attachable to the leg about a tibia of the leg, the tibia having a proximal end and a distal end, the tibial frame attachable to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom and output tibial motion data associated with the measured motion, wherein the femoral motion data and the tibial motion data are usable, in conjunction, to determine arthrokinematics in real-time.

2. A joint arthrometer in accordance with claim 1, wherein the connecting portion is curved to extend over the anterior side of the distal femur to accommodate the leg.

3. A joint arthrometer in accordance with claim 2, wherein the connecting portion is sprung to bias the first arm portion toward the medial side of the leg and bias the second arm portion toward the lateral side of the leg such that the femoral frame is capable of being clamped to the leg.

4. A joint arthrometer in accordance with claim 3, wherein the femoral frame is U-shaped.

5. A joint arthrometer in accordance with claim 1, wherein the tibial frame further comprises a placement guide, the placement guide extending proximally and distally from the rigid frame portion and configured to guide a user in placing the tibial frame.

6. A joint arthrometer in accordance with claim 1, wherein the arthrokinematics comprise tibiofemoral kinematics.

7. A joint arthrometer in accordance with claim 1, wherein during testing of the test subject when the test subject is wearing an orthotic in addition to the joint arthrometer, the femoral motion data and the tibial motion data comprise data that allows for an influence of the orthotic on knee motion of the test subject to be assessed.

8. A joint arthrometer in accordance with claim 1, the femoral motion sensor coupled to the connecting portion of the femoral frame.

9. A joint arthrometer in accordance with claim 1, the tibial frame being positionable over an anterior crest of the tibia.

10. A method for measuring arthrokinematics and muscle properties comprising:
attaching a femoral frame to a leg of a test subject about a distal femur of the leg, the distal femur having a medial side, a lateral side, an anterior side, a posterior side, a lateral epicondyle, and a medial epicondyle, the femoral frame comprising a first arm portion, a second arm portion, a connecting portion, a femoral motion sensor, and a femoral securing mechanism, the first arm portion contacting the leg about the lateral epicondyle, the second arm portion contacting the leg about the medial epicondyle, the connecting portion connecting the first and second arm portions, the connecting portion positioned above the anterior side of the leg, the femoral motion sensor coupled to the femoral frame and configured to measure motion with six degrees of freedom;
attaching a tibial frame to the leg about a tibia of the leg, the tibia having a proximal end and a distal end, the tibial frame attached to the leg about the tibia closer to the proximal end of the tibia than the distal end of the tibia, the tibial frame comprising a tibial securing mechanism, a rigid frame portion, and a tibial motion sensor, the tibial motion sensor coupled to the rigid frame portion and configured to measure motion with six degrees of freedom, the tibial securing mechanism attached to the rigid frame portion and positioned about the leg and tibia;
moving the tibia relative to the femur in at least one cycle of a functional task;
recording the motion of the femoral motion sensor and recording the motion of the tibial motion sensor through the at least one cycle; and
determining arthrokinematics based on the recorded motion of the femoral and tibial motion sensors.

11. A method in accordance with claim 10, wherein the determining of the arthrokinematics is based on calculations defined by a Cartesian coordinate system.

12. A method in accordance with claim 10, wherein the determining of the arthrokinematics is based on calculations defined by an orthogonal coordinate system.

13. A method in accordance with claim 10, wherein the at least one cycle includes at least one of knee flexion and extension, internal-external knee movement, and adduction-abduction knee movement.

14. A method in accordance with claim 10, wherein the functional task includes the test subject performing step-up movements on a step-up box.

15. A method in accordance with claim 10, further comprising attaching an auxiliary sensor to at least one of a malleoli or a calcaneus of the leg, recording a position of the auxiliary sensor, and determining spastic properties of the leg based on the position of the auxiliary sensor.

16. A method for measuring joint reaction comprising:
attaching a first sensor to a first portion of a leg of a test subject, the first sensor configured to measure a position of the first portion of the leg;
attaching a second sensor to a second portion of the leg different from the first portion of the leg, the second sensor configured to measure a position of the second portion of the leg;
obtaining and storing position data from the first sensor, the position data of the first sensor corresponding to the position of the first portion of the leg;
obtaining and storing position data from the second sensor, the position data of the second sensor corresponding to the position of the second portion of the leg;
performing a joint movement and storing at least one of a first sensor output of the first sensor resulting from the joint movement and a second sensor output of the second sensor resulting from the joint movement; and
determining a joint reaction response due to the joint movement, the joint reaction response being based on at least one of the first sensor output and the second sensor output.

17. A method in accordance with claim 16, wherein the first portion of the leg corresponds to a femur portion of the leg, the second portion of the leg corresponds to a tibia portion of the leg, and the joint movement comprises moving the tibia portion relative to the femur portion.

18. A method in accordance with claim 17, the leg further comprising a third portion, the method further comprising attaching a third sensor to the third portion of the leg, the third portion comprising at least one of a malleoli portion and a calcaneus portion of the leg, and the third sensor configured to output a third sensor output corresponding to position data of the third portion.

19. A method in accordance with claim 18, wherein the third sensor output comprises at least one of malleoli position data and calcaneus position data, and the performing of the joint movement comprises:
attaching the first sensor to the femur portion;
attaching the second sensor to the tibia portion;
attaching the third sensor to the malleoli portion in order to be able to obtain the malleoli position data;
after a pre-determined amount of time, obtaining and storing the malleoli position data;
attaching the third sensor to the calcaneus portion in order to be able to obtain the calcaneus position data and placing a foot of the leg in a first position;
after a pre-determined amount of time, obtaining and storing the calcaneus position data;
moving the tibia portion relative to the calcaneus portion to obtain spastic assessment data, the spastic assessment data corresponding to the joint reaction response; and
determining spastic properties of the leg based on the spastic assessment data.

20. A method in accordance with claim 19, wherein the malleoli portion comprises a lateral malleoli portion and a medial malleoli portion, and the attaching of the third sensor to the malleoli portion comprises:
attaching the third sensor to the lateral malleoli so as to be able to obtain and store lateral malleoli position data; and
attaching the third sensor to the medial malleoli so as to be able to obtain and store medial malleoli position data; and
the spastic properties include at least one of displacement properties corresponding to the joint reaction response, joint angle properties corresponding to the joint reaction response, and velocity properties corresponding to the joint reaction response.
